(19) United States
(12) Patent Application Publication
(10) Pub. No.: US 2024/0174627 A1
(43) Pub. Date: May 30, 2024

(54) CATALYTIC CANNABINOL SYNTHESIS AND PRECURSORS

(71) Applicant: KARE CHEMICAL TECHNOLOGIES INC., Mississauga (CA)

(72) Inventors: Kamaluddin ABDUR-RASHID, Mississauga (CA); Kareem ABDUR-RASHID, Mississauga (CA); Wenli JIA, Toronto (CA)

(73) Assignee: KARE CHEMICAL TECHNOLOGIES INC., Mississauga, ON (CA)

(21) Appl. No.: 18/549,719

(22) PCT Filed: Mar. 4, 2022
(86) PCT No.: PCT/CA2022/050311

§ 371 Date: Sep. 8, 2023

Related U.S. Application Data
(60) Provisional application No. 63/159,568, filed on Mar. 11, 2021.

(51) Int. Cl. C07D 311/80

(52) U.S. Cl. CPC C07D 311/80

(57) ABSTRACT

Cannabinol (CBN) is a minor cannabinoid found in the cannabis plant and there is significant interest in exploring CBN for pharmaceutical applications. It is currently being investigated for treating glaucoma, sleep disorders and skin inflammations. Several synthetic approaches for CBN have been described in the prior art, however methods are still limited in scope because of the harsh reaction conditions, number of steps involved, low yields and extensive purifications that are generally required. The present disclosure relates to new cannabinol precursor compounds and processes to prepare cannabinol compounds. The disclosure also relates to the use of catalysts and catalytic processes for the preparation of cannabinol compounds from the cannabinol precursors.

### RELATED APPLICATIONS

This application claims the benefit of U.S. Provisional Application No. 63/159,568 filed Mar. 11, 2021, the entire contents of which are hereby incorporated by reference.

### FIELD OF THE DISCLOSURE

The present disclosure relates to cannabinol precursor compounds and the use of the compounds for the preparation of cannabinol and its analogues. The disclosure also relates to the use of catalysts and catalytic processes for the preparation of cannabinol and its analogues using the cannabinol precursor compounds.

### BACKGROUND OF THE DISCLOSURE

Cannabinol (CBN) is a minor cannabinoid found in the cannabis plant. It has been reported to be non-psychoactive or mildly psychoactive. CBN is usually extracted from aged cannabis and the primary route is the oxidation of tetrahydrocannabinol (THC) facilitated by light and elevated temperatures during storage of harvested cannabis. It is not feasible to economically extract CBN directly from the plant for pharmaceutical use.

CBN is chemically more stable than CBD and THC under external stresses, such as heat and light. This feature of CBN allows for longer shelf-life, which is an important pharmaceutical parameter. Hence, there is significant interest in exploring CBN for pharmaceutical applications. It is currently being investigated for treating glaucoma, sleep disorders, skin inflammations, among others.

The supply of pure CBN for pharmaceutical, nutraceutical and recreational products is complicated by the limited supply from natural sources, and the demand for the more abundant and primary components (CBD and THC) of the cannabis plant. In addition, extracted cannabis resin contains more than 150 cannabinoid products, in addition to terpenes and other compounds present in the plant. The isolation of pure CBN from extracted and aged cannabis resin is laborious, time consuming and results in only low yields. Hence, there is a demand for high purity, and commercially viable supplies of CBN.

Several synthetic approaches for CBN have been described in the prior art. These include the use of high-pressure Diels-Alder reactions to prepare 6,6-dialkyl-benzo[c]chromenes, including CBN (L. Minuti et al., J. Org. Chem. 2012, 77, 7923-7931). In another approach, Appendino and co-workers reported the use of iodine for the aromatization of CBD and THC (F. Pollastro et al., J. Nat. Prod. 2018, 81, 630-633). However, these and other methods are still limited in scope because of the harsh reaction conditions, number of steps, low yields and extensive purifications that are generally required.

In addition, there are essentially no practical routes for the isolation of the CBN derivatives of the rare cannabinoids THCV, THCB and THCP from the cannabis plants. Unlike THC, these analogues are either rare or only detectible in miniscule amounts in the cannabis plant.

The prior art reflects the difficulties associated with developing a reliable and commercially viable route for synthetic CBN and its analogues.

### SUMMARY OF THE DISCLOSURE

The present disclosure, in some aspects, describes a new approach to the synthesis of cannabinol and its analogues that focuses on the use of novel and stable precursors that can be transformed into the desired cannabinol product on demand. The precursors are derived from commercially available chemicals.

In various aspects, the disclosure relates to the preparation of new cannabinol precursor compounds for the preparation of cannabinol and its analogues and derivatives using catalysts and catalytic processes. The precursors can be prepared and purified prior to transformation to the desired cannabinol products. The precursors are air-stable and shelf-stable compounds that can be stored, transported and converted into the desired cannabinol products on demand.

Accordingly, in some embodiments, the present disclosure relates to cannabinol precursors of Formula (I):

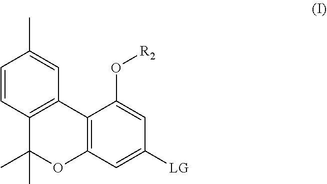

wherein,
LG is any suitable leaving group, and
R₂ represents hydrogen, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

In one embodiment, the leaving group is a halo group, a sulphonate, or a boronate.

In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₀)-aryl group. In another embodiment, the boronate leaving group is —B(OR)₂, where R is H, a (C₁-C₂₀)-alkyl group (such as a (C₁-C₁₀)-alkyl group) or a (C₆-C₁₀)-aryl group (such as (C₆-C₁₀)-aryl group). In another embodiment, the boronate leaving group is —BF₃K.

In another embodiment, the leaving group is a sulfonate is of the formula

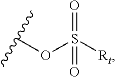

wherein,
Rₜ is a hydrogen atom, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an OR^{c} group or an NR^{c}₂ group, possibly substituted, with possible and non-limiting substituents of Rₜ being halogen atoms, OR^{c}, or NR^{c}₂ groups, in which R^{c} is a hydrogen atom or a cyclic, linear or branched alkyl, aryl or alkenyl group.

In one embodiment, the sulfonate leaving group is a triflate, mesylate or tosylate group.

In a general way, the compounds of Formula (I) can be prepared and isolated prior to use.

In some other aspects, the present disclosure relates to cannabinol sulfonate esters of Formula (II):

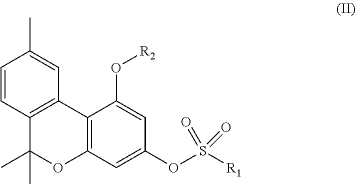

wherein,
R₁ represents a hydrogen atom, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, an OR^{c} group or an NR^{c}₂ group, possibly substituted, with possible and non-limiting substituents of R₁ being halogen atoms, OR^{d}, or NR^{d}₂ groups, in which R^{c} or R^{d} is a hydrogen atom or a cyclic, linear or branched alkyl, aryl or alkenyl group; and
R₂ represents a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

In a general way, the compounds of Formula (II) can be prepared and isolated prior to use.

In some embodiments, the present disclosure relates to cannabinol sulfonate esters of Formula (III):

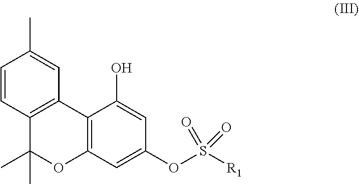

wherein,
R₁ represents a hydrogen atom, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, an OR^{c} group or an NR^{c}₂ group, possibly substituted, with possible and non-limiting substituents of R₁ being halogen atoms, OR^{d}, or NR^{d}₂ groups, in which R^{c} or R^{d} is a hydrogen atom or a cyclic, linear or branched alkyl, aryl or alkenyl group.

In a general way, the compounds of Formula (III) can be prepared and isolated prior to use.

In some other aspects, the present disclosure also relates to cannabinol precursors of Formula (IV):

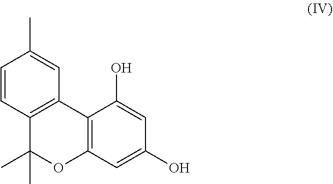 [SMILES: Cc1ccc2c(c1)-c1c(O)cc(O)cc1OC2(C)C]

In some other aspects, the present disclosure also relates to cannabinol precursors of Formula (I) to Formula (IV), wherein one or more of the hydrogen atoms are replaced with deuterium.

In some other aspects, the present disclosure also relates to cannabinol precursors of Formula (I) to Formula (IV), wherein one or more of the carbon-12 atoms are replaced with carbon-13.

In another embodiment, the present disclosure relates to cannabinol precursors of Formula (V):

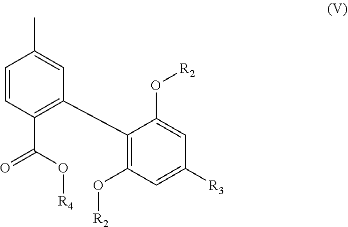

wherein,
one or more of the hydrogen atoms are replaced with deuterium and/or one or more of the carbon-12 atoms are replaced with carbon-13;
R₂ represents a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ and R₄ represents a hydrogen atom, a linear or branched alkyl group of any length, possibly substituted, an alkenyl group of any length, possibly substituted, an alkynyl group, possibly substituted, a cycloalkyl group, possibly substituted, or an aryl group, possibly substituted.

In another embodiment, the present disclosure relates to cannabinol precursors of Formula (VI):

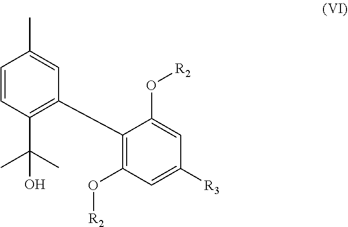

wherein,
one or more of the hydrogen atoms are replaced with deuterium and/or one or more of the carbon-12 atoms are replaced with carbon-13;
R₂ represents a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ represents a hydrogen atom, a linear or branched alkyl group of any length, possibly substituted, an alkenyl group of any length, possibly substituted, an alkynyl group, possibly substituted, a cycloalkyl group, possibly substituted, or an aryl group, possibly substituted.

In another embodiment, the present disclosure relates to cannabinol compounds of Formula (VII):

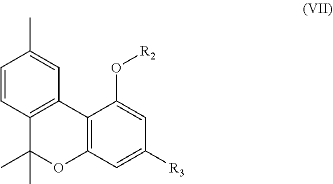

wherein,
one or more of the hydrogen atoms in the p-cymene fragment of the molecule are replaced with deuterium and/or one or more of the carbon-12 atoms in the p-cymene fragment of the molecule are replaced with carbon-13;
R₂ represents hydrogen, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ represents a hydrogen atom, a linear or branched alkyl group of any length, possibly substituted, an alkenyl group of any length, possibly substituted, an alkynyl group, possibly substituted, a cycloalkyl group, possibly substituted, or an aryl group, possibly substituted.

In various embodiments of the disclosure, the transformations to which the compounds of the disclosure can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the present disclosure to prepare one or more of the cannabinol compounds selected from the group consisting of:

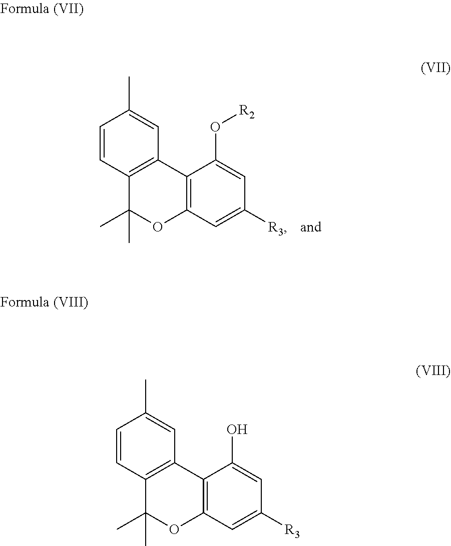

wherein,
R₂ represents a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ represents a hydrogen atom, a linear or branched alkyl group of any length, possibly substituted, an alkenyl group of any length, possibly substituted, an alkynyl group, possibly substituted, a cycloalkyl group, possibly substituted, or an aryl group, possibly substituted.

In some other aspects, the present disclosure also relates to the preparation of cannabinol compounds of Formula (VII) and Formula (VIII), wherein one or more of the hydrogen atoms are replaced with deuterium.

In some other aspects, the present disclosure also relates to the preparation of cannabinol compounds of Formula (VII) and Formula (VIII), wherein one or more of the carbon-12 atoms are replaced with carbon-13 atoms.

In some other aspects, the present disclosure also relates to the preparation of cannabinol compounds of Formula (VII) and Formula VIII), wherein one or more of the hydrogen atoms of the p-cymene fragment of the molecule are replaced with deuterium.

In some other aspects, the present disclosure also relates to the preparation of cannabinol compounds of Formula (VII) and Formula (VIII), wherein one or more of the carbon-12 atoms of the p-cymene fragment of the molecule are replaced with carbon-13 atoms.

In some other aspects of the disclosure, the present disclosure provides a method for the synthesis of one or more of the cannabinol products below:

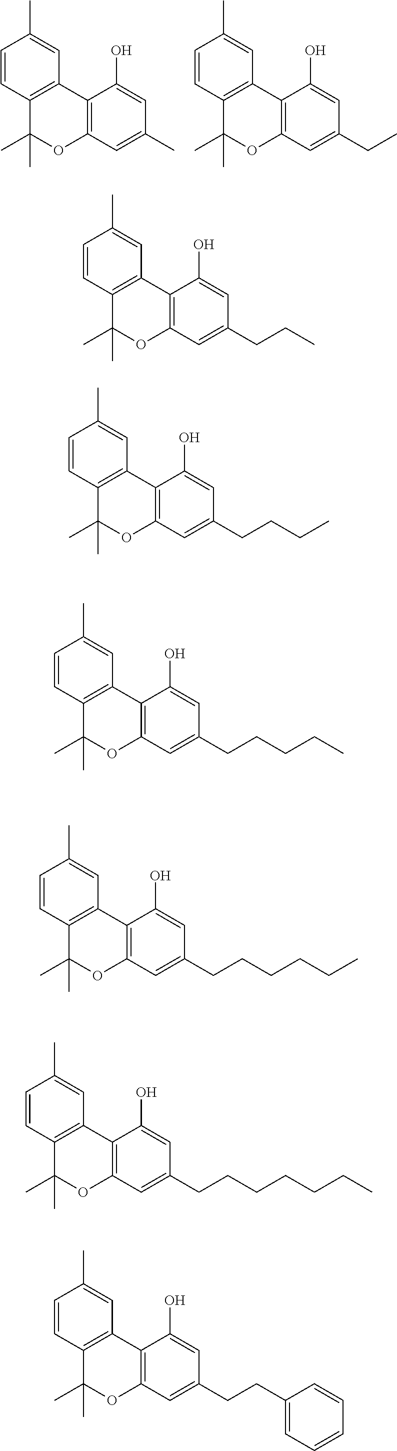

In some other aspects of the disclosure, the present disclosure provides a method for the synthesis of one or more of the deuterated cannabinol products below:

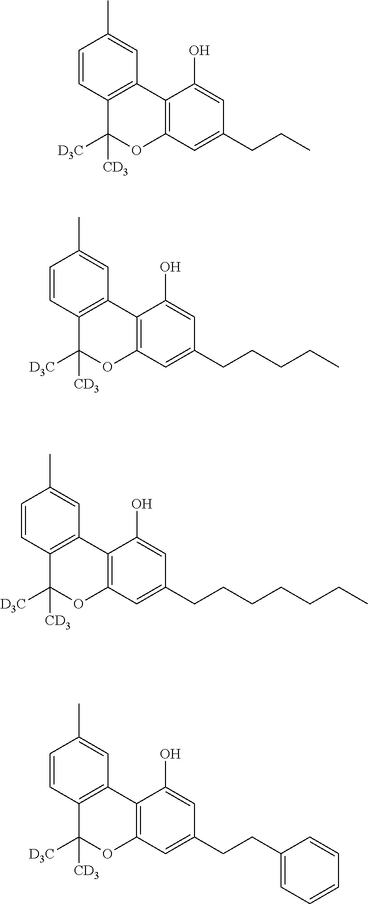 [SMILES: [2H]C([2H])([2H])C1(C([2H])([2H])[2H])Oc2cc(CCC)cc(O)c2-c2cc(C)ccc21 ; [2H]C([2H])([2H])C1(C([2H])([2H])[2H])Oc2cc(CCCCC)cc(O)c2-c2cc(C)ccc21 ; [2H]C([2H])([2H])C1(C([2H])([2H])[2H])Oc2cc(CCCCCCC)cc(O)c2-c2cc(C)ccc21 ; [2H]C([2H])([2H])C1(C([2H])([2H])[2H])Oc2cc(CCc3ccccc3)cc(O)c2-c2cc(C)ccc21]

In some other aspects of the disclosure, the present disclosure provides a method for the synthesis of one or more of the carbon-13 cannabinol products below:

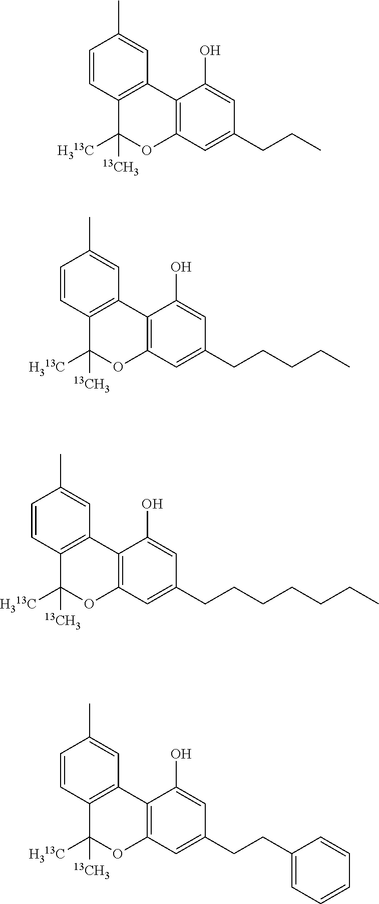 [SMILES: CCCc1cc(O)c2c(c1)OC([13CH3])([13CH3])c1ccc(C)cc1-2 ; CCCCCc1cc(O)c2c(c1)OC([13CH3])([13CH3])c1ccc(C)cc1-2 ; CCCCCCCc1cc(O)c2c(c1)OC([13CH3])([13CH3])c1ccc(C)cc1-2 ; Cc1ccc2c(c1)-c1c(O)cc(CCc3ccccc3)cc1OC2([13CH3])[13CH3]]

In some aspects the disclosure provides a process for the catalytic preparation of compounds of Formula (VII) and Formula (VIII).

In some other aspects the disclosure provides a process for the non-catalytic preparation of compounds of Formula (VII) and Formula (VIII) from compounds of Formula (I) to Formula (VI).

In various embodiments, the process for the preparation of compounds of Formula (VII) and Formula (VIII) from compounds of Formula (I) to Formula (VI), pursuant to the disclosure uses a boron containing compound such as R₃—B(OH)₂, R₃—B(OR)₂ or R₃—BF₃K.

In some other aspects of the process of the disclosure a Grignard compound such as R₃—MgX is used to prepare compounds of Formula (VII) and Formula (VIII).

In still other aspects of the process of the disclosure an organozinc compound such as R₃—ZnX is used to prepare compounds of Formula (VII) and Formula (VIII).

The present disclosure also includes, compositions, methods of producing the compounds and compositions comprising the compounds of the disclosure, kits comprising any one or more of the components of the foregoing, optionally with instructions to make or use same and uses of any of the foregoing.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will be described in greater detail with reference to the following drawings in which, which are meant to be illustrative by certain embodiments of the disclosure and are not meant to limit the scope of the disclosure:

FIG. 1 shows the scheme for the preparation of cannabinol (CBN) in one embodiment of the disclosure;

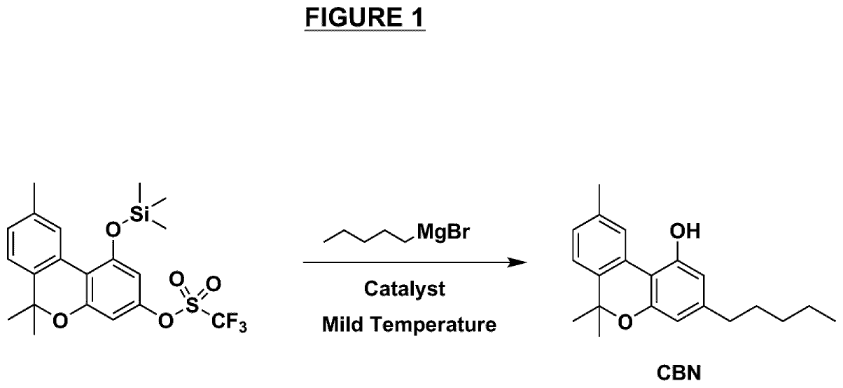

FIG. 2 shows the X-ray crystal structure of methyl 2′,4′,6′-trimethoxy-5-methylbiphenyl-2-carboxylate in one embodiment of the disclosure;

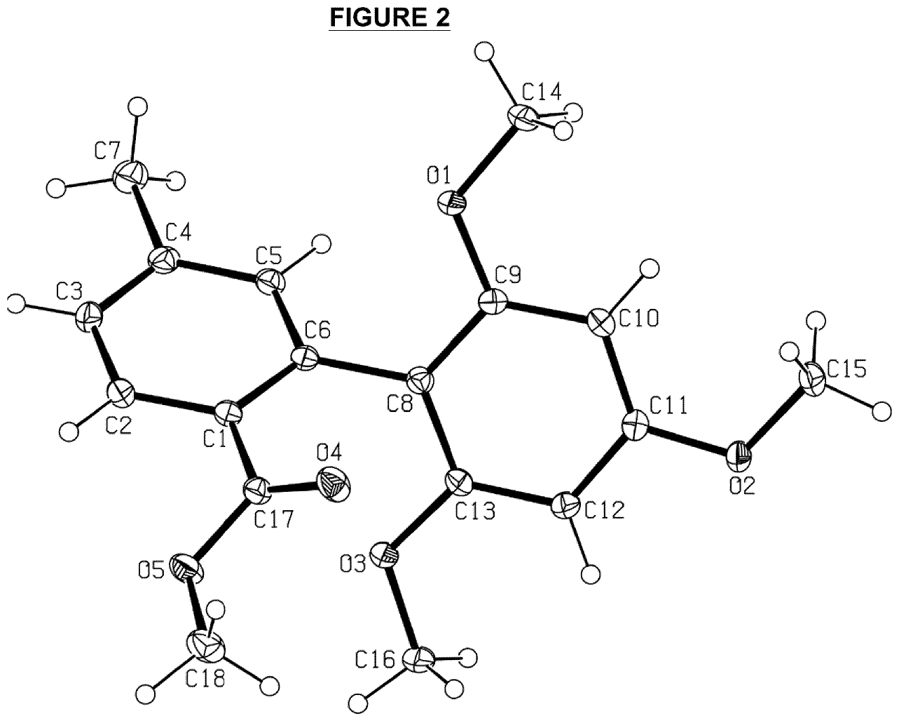

FIG. 3 shows the X-ray crystal structure of 2-(2′,4′,6′-trimethoxy-5-methylbiphenyl-2-yl)propan-2-ol in one embodiment of the disclosure;

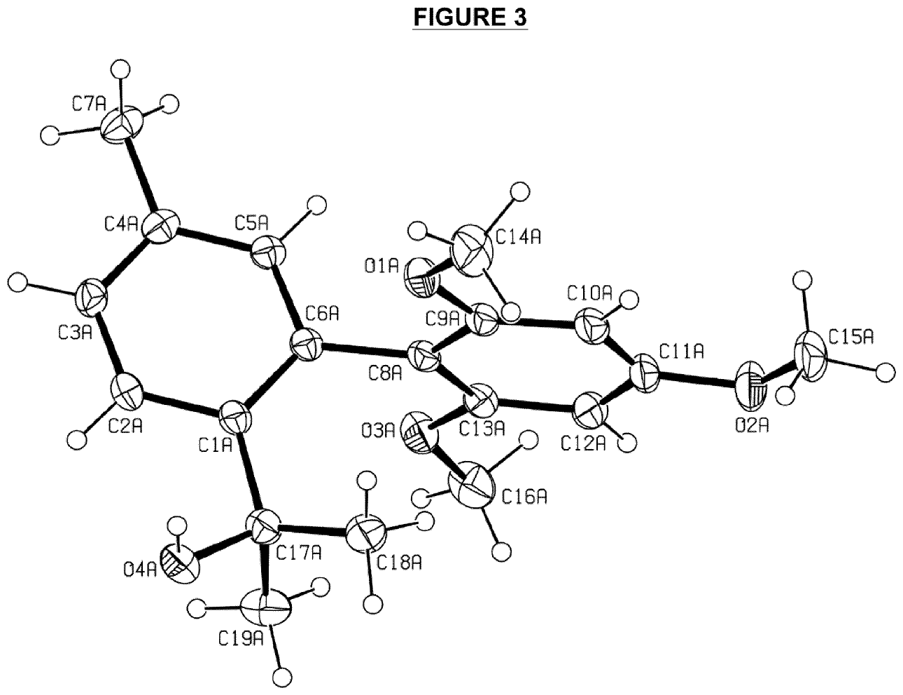

FIG. 4 shows the X-ray crystal structure of 6,6,9-trimethyl-6H-benzo[c]chromene-1,3-diol in one embodiment of the disclosure;

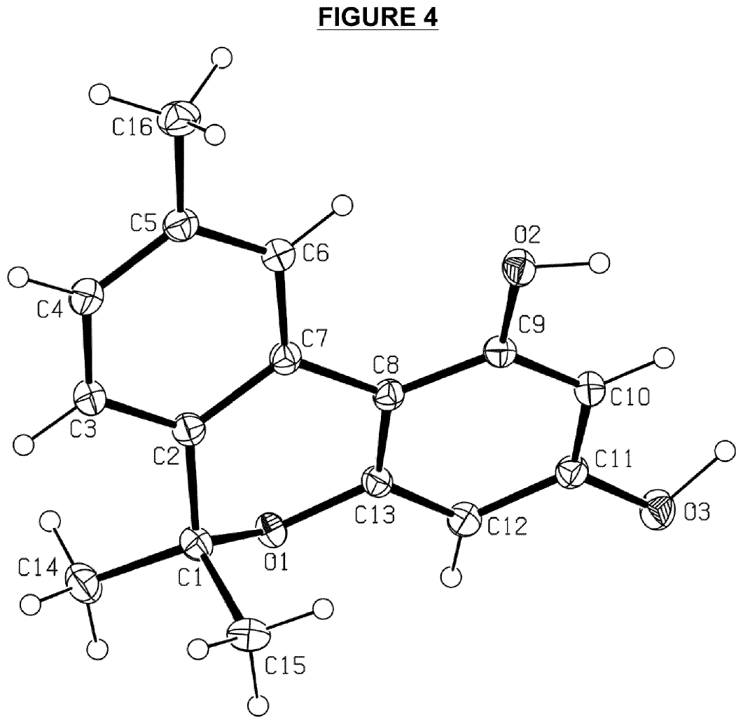

FIG. 5 shows the X-ray crystal structure of 1-hydroxy-6,6,9-trimethyl-6H-benzo[c]chromen-3-yl trifluoromethanesulfonate in one embodiment of the disclosure;

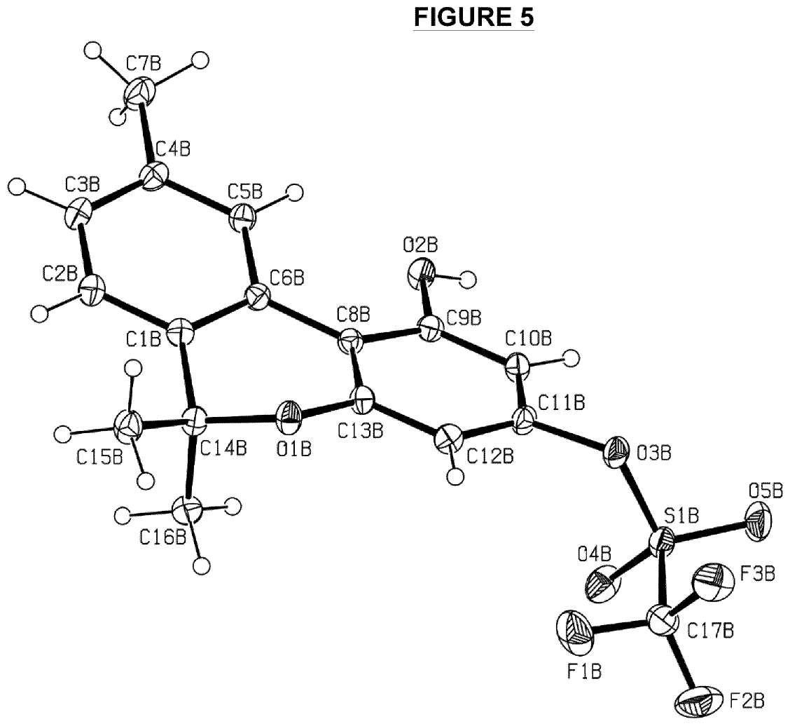

FIG. 6 shows the ¹H NMR spectrum of methyl 2′,4′,6′-trimethoxy-5-methylbiphenyl-2-carboxylate in one embodiment of the disclosure;

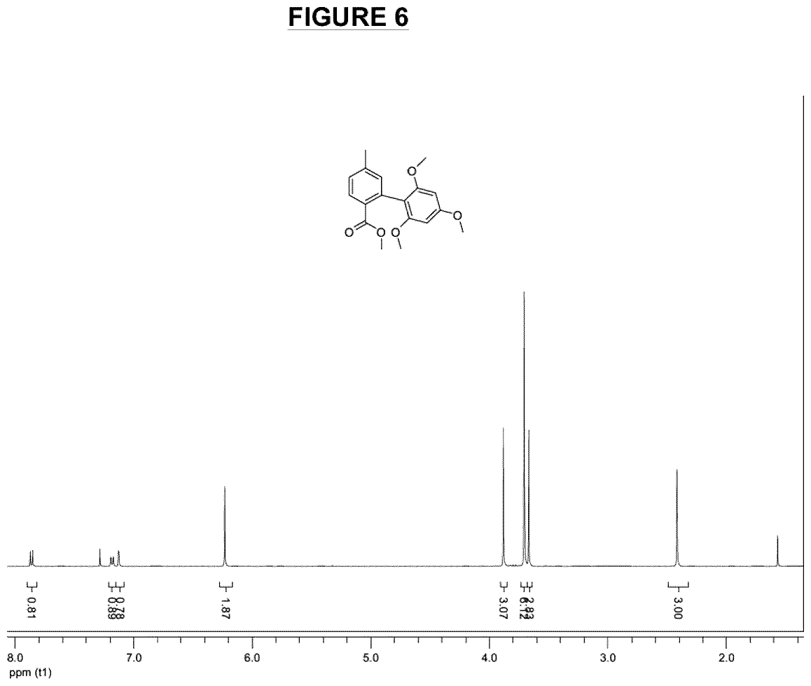

FIG. 7 shows the ¹H NMR spectrum of 2-(2′,4′,6′-trimethoxy-5-methylbiphenyl-2-yl)propan-2-ol in one embodiment of the disclosure;

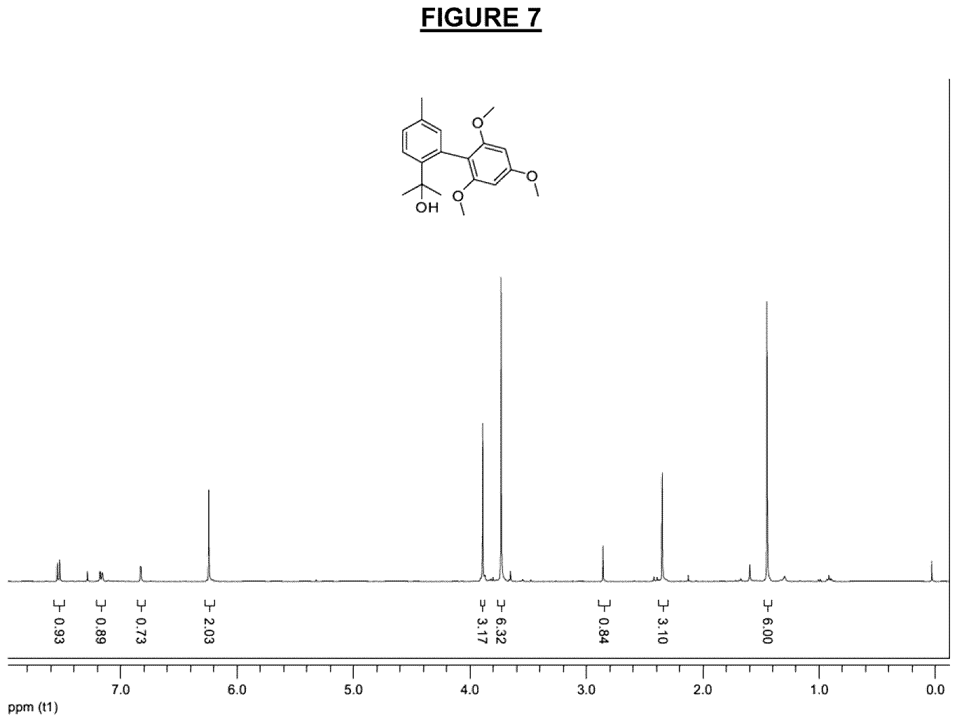

FIG. 8 shows the ¹H NMR spectrum of 6,6,9-trimethyl-6H-benzo[c]chromene-1,3-diol in one embodiment of the disclosure;

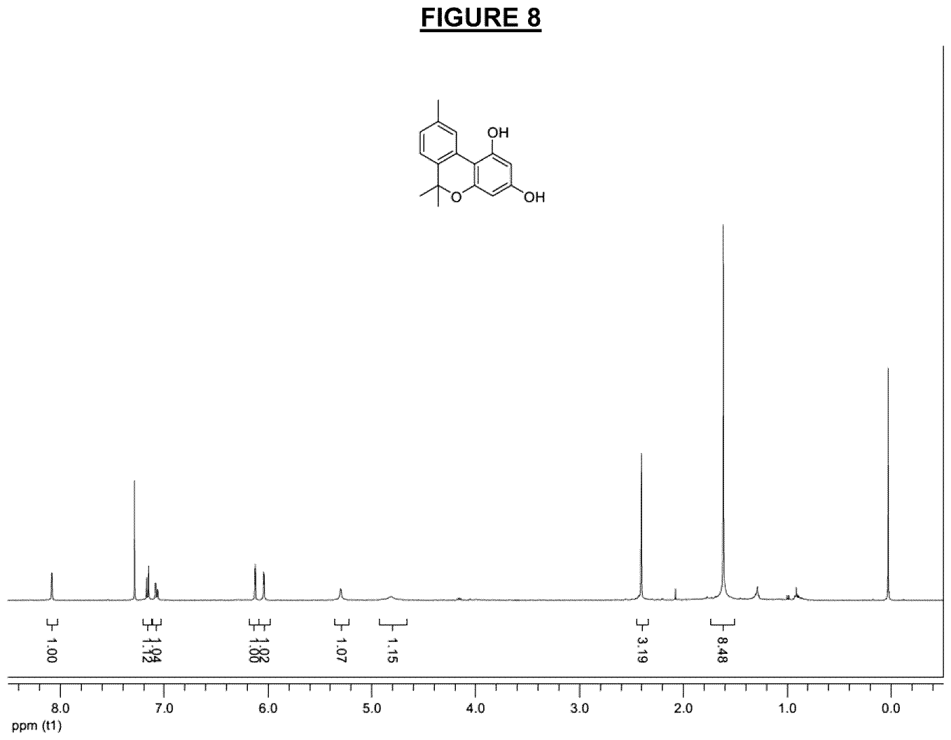

FIG. 9 shows the ¹H NMR spectrum of 1-hydroxy-6,6,9-trimethyl-6H-benzo[c]chromen-3-yl trifluoromethanesulfonate in one embodiment of the disclosure;

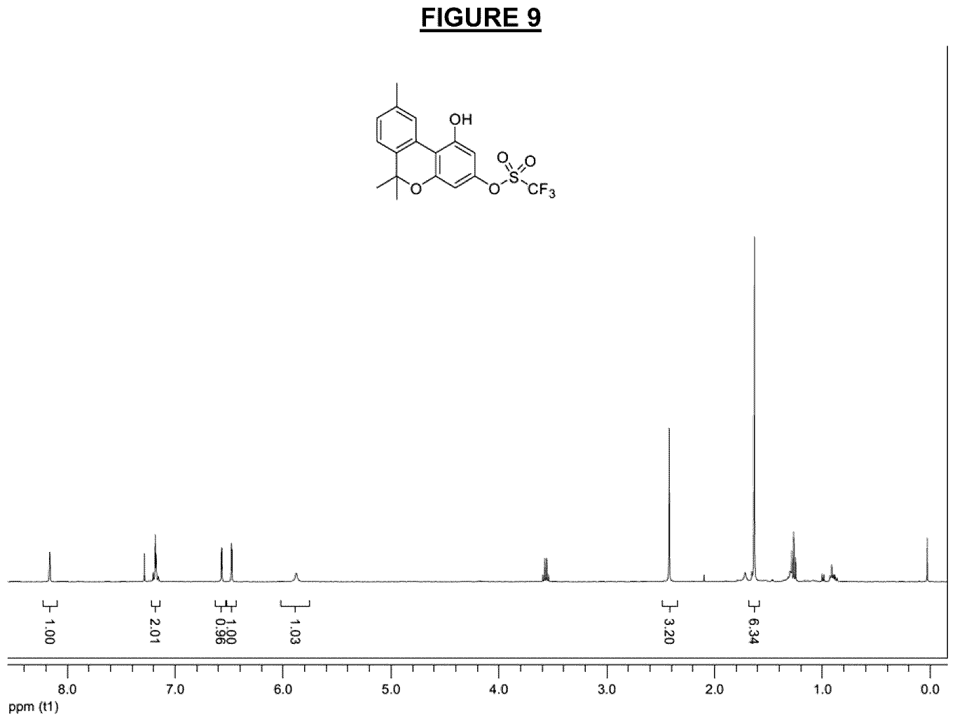

FIG. 10 shows the ¹H NMR spectrum of 6,6,9-trimethyl-1-(trimethylsilyloxy)-6H-benzo[c]chromen-3-yl trifluoromethanesulfonate in one embodiment of the disclosure;

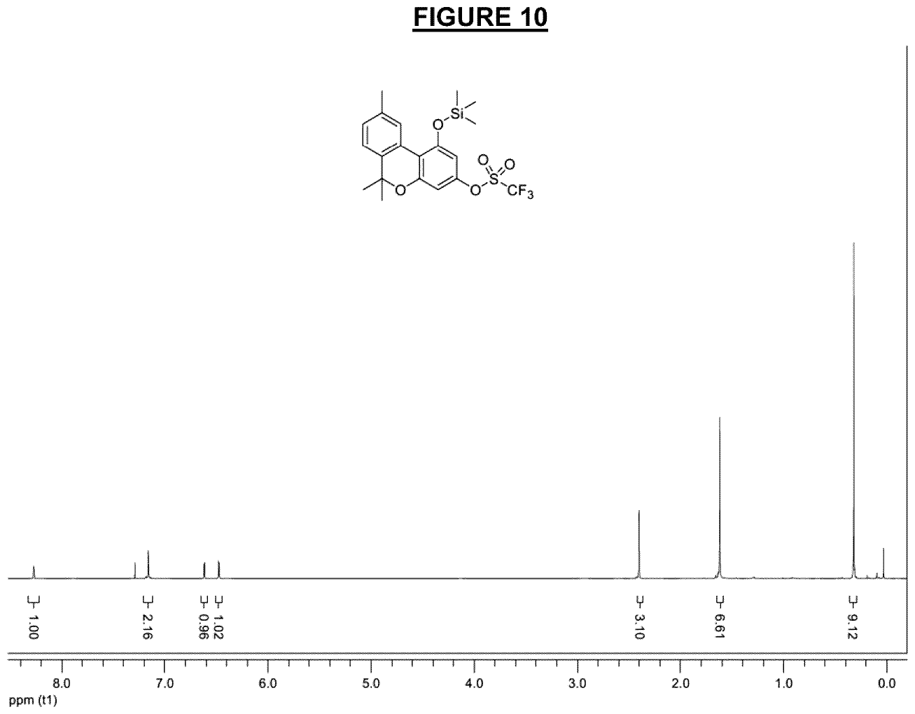

FIG. 11 shows the ¹H NMR spectrum of Cannabinol-C1 (CBN-C1) in one embodiment of the disclosure;

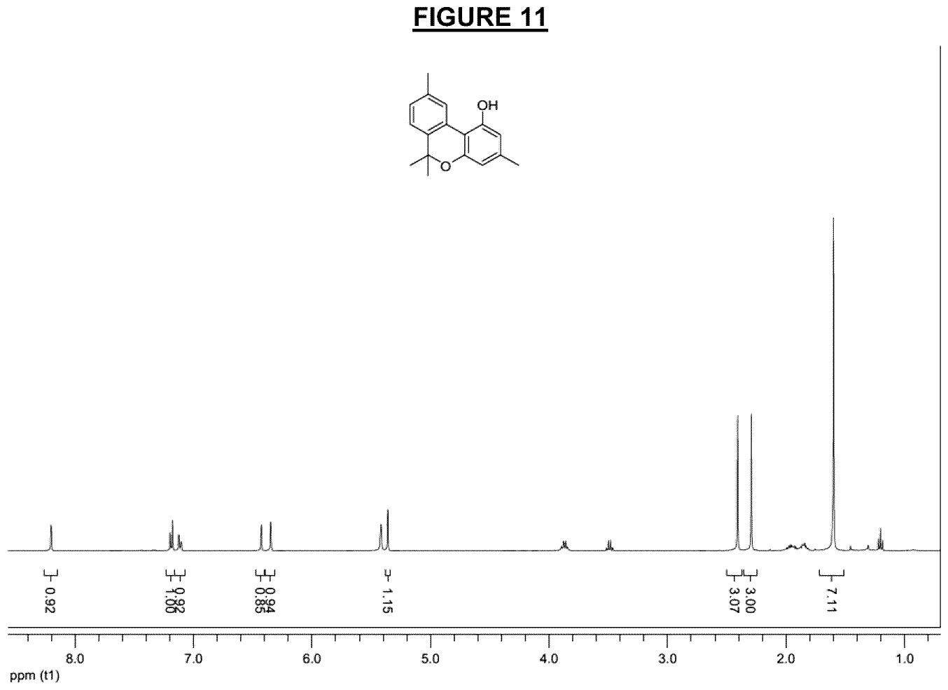

FIG. 12 shows the ¹H NMR spectrum of Cannabinol-C2 (CBN-C2) in one embodiment of the disclosure;

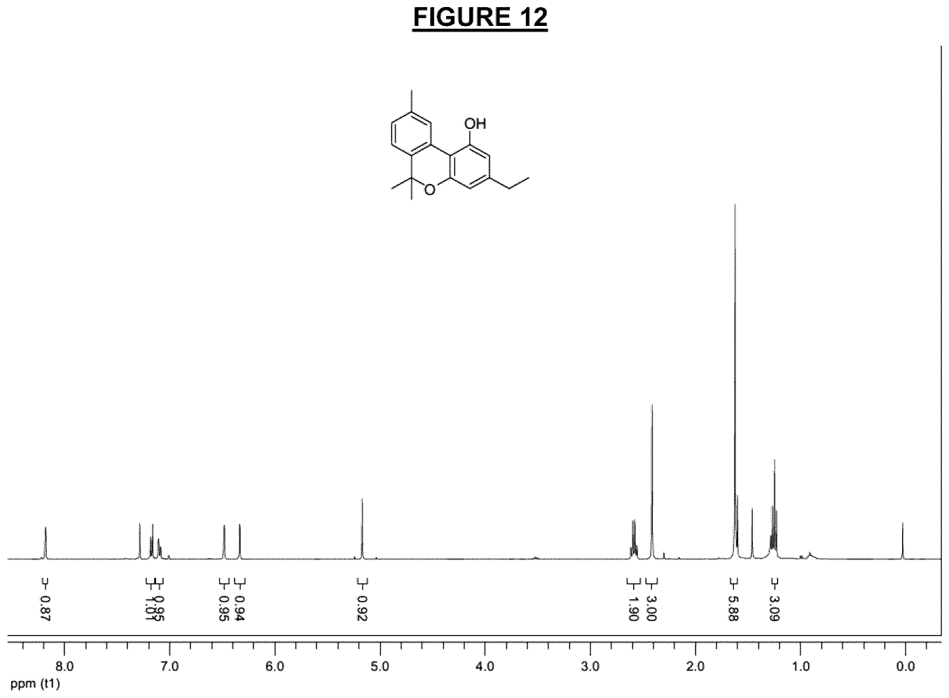

FIG. 13 shows the ¹H NMR spectrum of Cannabivarin (CBNV) in one embodiment of the disclosure;

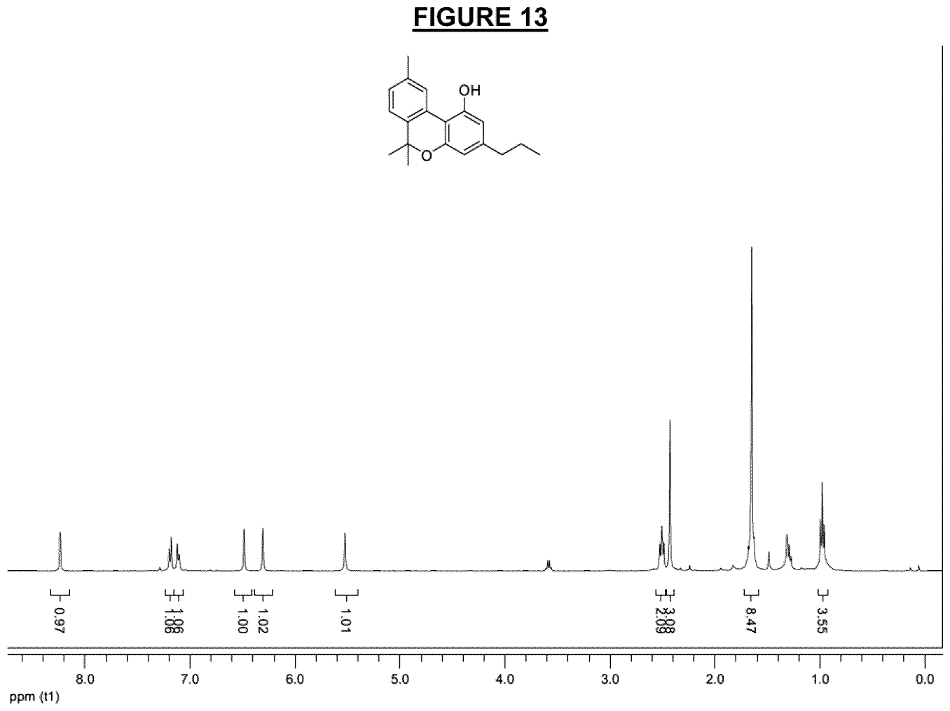

FIG. 14 shows the ¹H NMR spectrum of Cannabibutol (CBNB) in one embodiment of the disclosure;

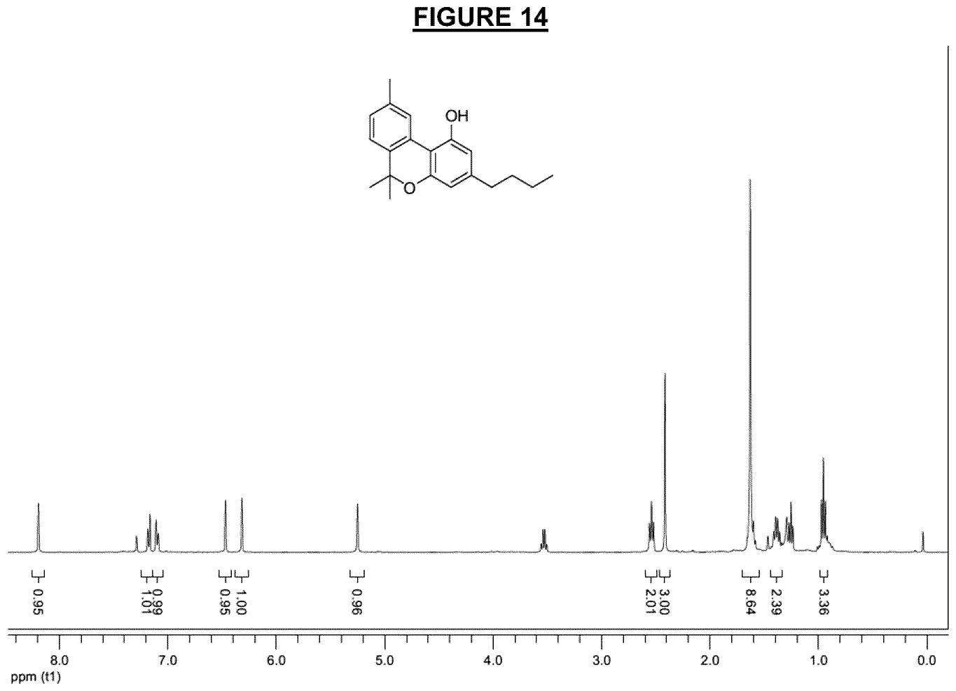

FIG. 15 shows the ¹H NMR spectrum of Cannabinol (CBN) in one embodiment of the disclosure;

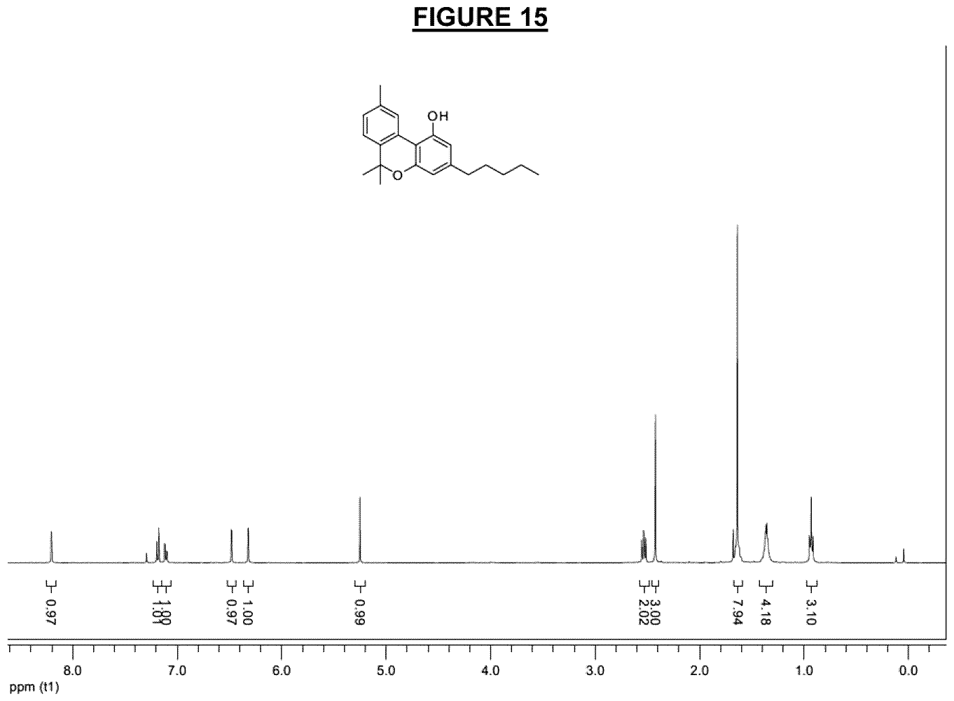

FIG. 16 shows the ¹H NMR spectrum of Cannabihexol (CBNH) in one embodiment of the disclosure;

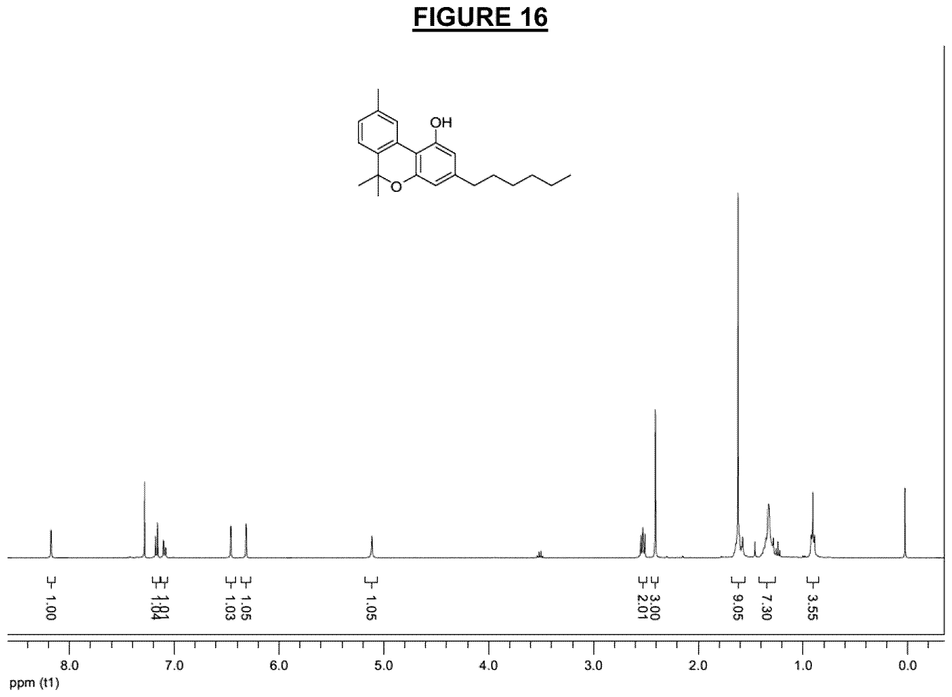

FIG. 17 shows the ¹H NMR spectrum of Cannabiphorol (CBNP) in one embodiment of the disclosure;

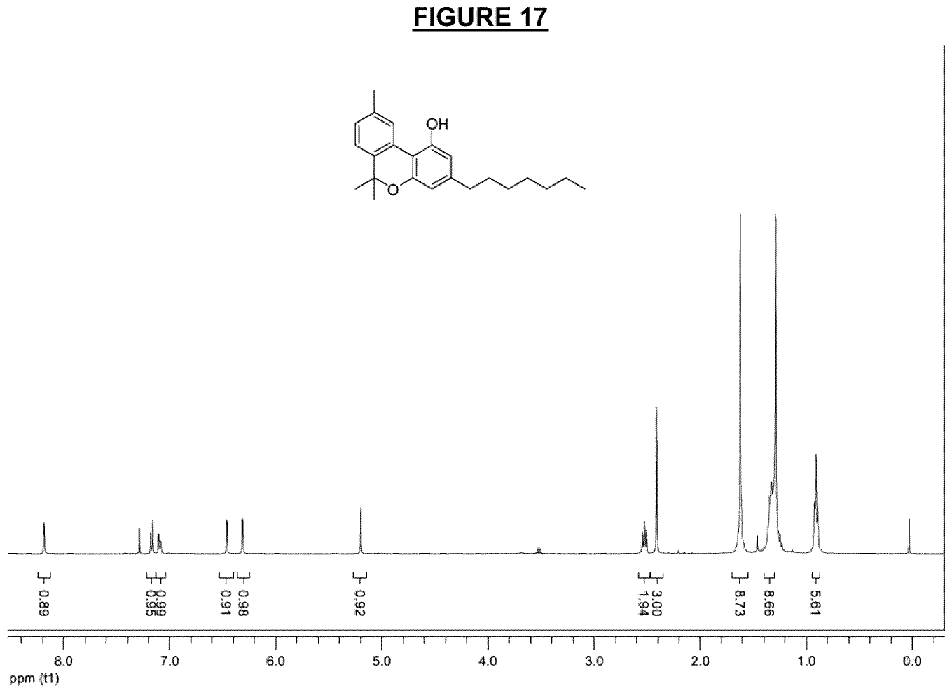

FIG. 18 shows the ¹H NMR spectrum of Benzyl-Cannabinol in one embodiment of the disclosure.

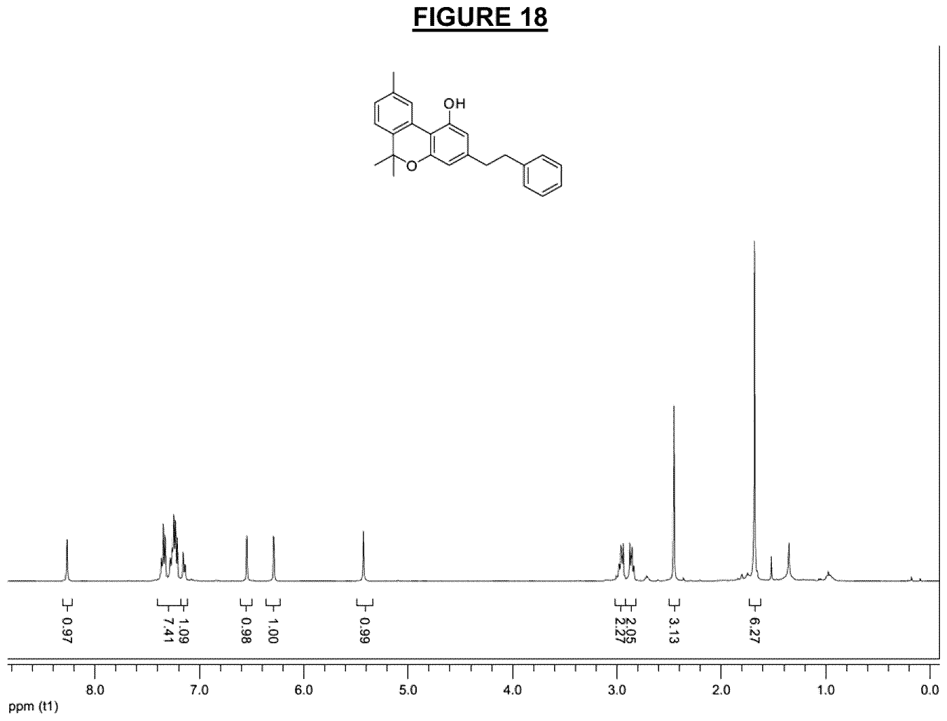

### DETAILED DESCRIPTION OF THE DISCLOSURE

### (I) Definitions

The term “(C₁-Cₘ)-alkyl” as used herein means straight and/or branched chain, saturated alkyl radicals containing one or more carbon atoms and includes (depending on the identity of “m”) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable “m” denotes the largest number of carbon atoms.

The term “(C₂-Cₘ)-alkenyl” as used herein means straight and/or branched chain, unsaturated alkyl radicals containing two or more carbon atoms and one to three double bonds, and includes (depending on the identity “m”) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable “m” denotes the largest number of carbon atoms.

The term “(C₁-Cₘ)-alkynyl” as used herein means straight and/or branched chain, unsaturated alkyl radicals containing two or more carbon atoms and one to three triple bonds, and includes (depending on the identity of “m”) acetylynyl, propynyl, but-1-ynyl, but-2-ynyl, but-3-ynyl, 3-methylbut-1-enyl, 3-methylpent-1-ynyl, 4-methylpent-1-ynyl, 4-methylpent-2-ynyl, penta-1,3-di-ynyl, hexyn-1-yl and the like, where the variable “m” denotes the largest number of carbon atoms.

The term “alkoxy” as used herein means straight and/or branched chain alkoxy group containing one or more carbon atoms and includes (depending on the identity) methoxy, ethoxy, propyloxy, isopropyloxy, t-butoxy, heptoxy, and the like.

The term “(C₃-Cₘ)-cycloalkyl” as used herein means a monocyclic, bicyclic or tricyclic saturated carbocylic group containing three or more carbon atoms and includes (depending on the identity of “m”) cyclopropyl, cyclobutyl, cyclopentyl, cyclodecyl and the like, where the variable “m” denotes the largest number of carbon atoms.

The term “(C₆-Cₘ)-aryl” as used herein means a monocyclic, bicyclic or tricyclic aromatic ring system containing at least one aromatic ring and 6 or more carbon atoms and includes phenyl, naphthyl, anthracenyl, 1,2-dihydronaphthyl, 1,2,3,4-tetrahydronaphthyl, fluorenyl, indanyl, indenyl and the like, where the variable “m” denotes the largest number of carbon atoms

The term “(C₅-Cₘ)-heteroaryl” as used herein means a monocyclic, bicyclic or tricyclic ring system containing one or two aromatic rings and 5 or more atoms of which, unless otherwise specified, one, two, three, four or five are heteromoieties independently selected from N, NH, N(alkyl), O and S and includes thienyl, furyl, pyrrolyl, pyrididyl, indolyl, quinolyl, isoquinolyl, tetrahydroquinolyl, benzofuryl, benzothienyl and the like, where the variable “m” denotes the largest number of carbon atoms.

The term “leaving group” or “LG” as used herein refers to a group that is readily displaceable by a nucleophile, for example, under nucleophilic substitution reaction conditions.

The term “halo” or “halogen” as used herein means chloro, fluoro, bromo or iodo.

The term “fluoro-substituted” as used herein means that at least one, including all, of the hydrogens on the referenced group is replaced with fluorine.

The suffix “ene” added on to any of the above groups means that the group is divalent, i.e. inserted between two other groups.

The term “ring system” as used herein refers to a carbon-containing ring system, that includes monocycles, fused bicyclic and polycyclic rings, bridged rings and metalocenes. Where specified, the carbons in the rings may be substituted or replaced with heteroatoms.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. For instance, “including” also encompasses “including but not limited to”. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Compounds of the Disclosure

The present disclosure relates to cannabinol precursors of Formula (I):

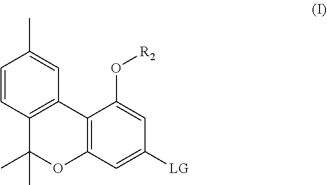

wherein
LG is any suitable leaving group, and
R₂ represents hydrogen, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

In one embodiment, LG is an anionic group (after leaving) such as sulphonates, halides, boronates; or MXₙ groups (M=Li, Mg, Zn, Sn, B, Si; X is halide, OH, OR, (wherein R is (C₁-C₂₀)-alkyl, (C₂-C₂₀)-alkenyl group, a (C₂-C₂₀)-alkynyl group, a (C₃-C₂₀)-cycloalkyl group, or (C₆-C₂₀)-aryl; n=0 to 3). In another embodiment, the (C₁-C₂₀)-alkyl is (C₁-C₁₀)-alkyl, or (C₁-C₆)-alkyl, the (C₂-C₂₀)-alkenyl group is (C₂-C₁₀)-alkenyl group or (C₂-C₆)-alkenyl group; the (C₂-C₂₀)-alkynyl group is (C₂-C₁₀)-alkynyl group or (C₂-C₆)-alkynyl group; the (C₃-C₂₀)-cycloalkyl group is (C₃-C₁₀)-cycloalkyl group or (C₃-C₆)-cycloalkyl group; and the (C₆-C₂₀)-aryl is (C₆-C₁₀)-aryl or (C₆-C₁₀)-aryl or (C₆)-aryl.

In one embodiment, the leaving group is a halo group, a sulphonate, or a boronate.

In another embodiment, the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₀)-aryl group. In another embodiment, the boronate leaving group is —B(OR)₂, where R is H, a (C₁-C₂₀)-alkyl group (such as a (C₁-C₁₀)-alkyl group or (C₁-C₆)-alkyl group) or a (C₆-C₁₄)-aryl group (such as a (C₆-C₁₀)-aryl group or (C₆)-aryl).

In another embodiment, the boronate leaving group is —BF₃K.

In another embodiment, the leaving group is a sulfonate is of the formula

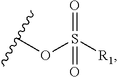

wherein,
Rₜ is a hydrogen atom, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an OR^{c} group or an NR^{c}₂ group, possibly substituted, with possible and non-limiting substituents of Rₜ being halogen atoms, OR^{c}, or NR^{c}₂ groups, in which R^{c} is a hydrogen atom or a cyclic, linear or branched alkyl, aryl or alkenyl group.

In another embodiment, Rₜ is a hydrogen atom, an optionally substituted (C₁-C₂₀)-alkyl group, an optionally substituted (C₂-C₂₀)-alkenyl group, an optionally substituted (C₂-C₂₀)-alkynyl group, an optionally substituted (C₃-C₂₀)-cycloalkyl group, an optionally substituted (C₆-C₁₄)-aryl group, an optionally substituted (C₅-C₁₄)-heteroaryl group, an OR^{c} group or an NR^{c}₂ group, wherein the optional substituents are halogen atoms, OR^{d}, or NR^{d}₂ groups, in which R^{c} or R^{d} is a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group.

In another embodiment, Rₜ is a hydrogen atom, an optionally substituted (C₁-C₁₀)-alkyl group, an optionally substituted (C₂-C₁₀)-alkenyl group, an optionally substituted (C₂-C₁₀)-alkynyl group, an optionally substituted (C₃-C₁₀)-cycloalkyl group, an optionally substituted (C₆-C₁₀)-aryl group. In another embodiment, Rₜ is a hydrogen atom, an optionally substituted (C₁-C₆)-alkyl group, an optionally substituted (C₂-C₆)-alkenyl group, an optionally substituted (C₂-C₆)-alkynyl group, an optionally substituted (C₃-C₆)-cycloalkyl group, an optionally substituted (C₆)-aryl group. In another embodiment, any of the alkyl groups are fluoro-substituted, for example, a fluoro-substituted (C₁-C₆)-alkyl group, such as CF₃.

In another embodiment, the optional substituents are, a (C₁-C₁₀)-alkyl group, a (C₂-C₁₀)-alkenyl group, a (C₂-C₁₀)-alkynyl group, a (C₃-C₁₀)-cycloalkyl group, or a (C₆-C₁₀)-aryl group. In another embodiment, a (C₁-C₆)-alkyl group, a (C₂-C₆)-alkenyl group, a (C₂-C₆)-alkynyl group, a (C₃-C₆)-cycloalkyl group, or a (C₆)-aryl group.

In one embodiment, the sulfonate leaving group is a triflate, mesylate or tosylate group.

In one embodiment, R₂ represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₀)-aryl group, or a (C₆-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a (C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

In one embodiment, R₂ represent a (C₁-C₁₀)-alkyl group, a (C₂-C₁₀)-alkenyl group, a (C₂-C₁₀)-alkynyl group, a (C₃-C₁₀)-cycloalkyl group, a —Si[C₁-C₁₀)-alkyl]₃ group, a (C₆-C₁₀)-aryl group, or a (C₅-C₁₀)-heteroaryl group, or an acyl group —C(═O)—R′, wherein R′ is a (C₁-C₁₀)-alkyl group, wherein each group is each optionally substituted with one or more halogen atoms (F, Cl, Br or I), a —(C₁-C₁₀)-alkyl group, a (C₂-C₁₀)-alkenyl group, a (C₂-C₁₀)-alkynyl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₁₀)-alkyl groups.

In one embodiment, R₂ represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms (F, Cl, Br or I), a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₆)-alkyl groups.

In one embodiment, R₂ represent a (C₁-C₆)-alkyl group, a —Si[(C₁-C₆)-alkyl]₃ group, or a phenyl group.

In one embodiment, R₂ represent a —Si[(C₁-C₆)-alkyl]₃ group. In one embodiment, R₂ represent a —Si[(C₁-C₃)-alkyl]₃ group. In one embodiment, R₂ represent a —Si(CH₃)₃ group.

The present disclosure also relates to cannabinol sulfonate esters of Formula (II):

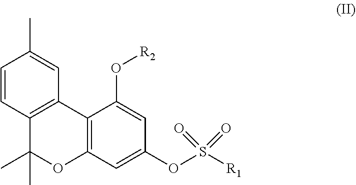

wherein,
R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group; and
R₂ is as defined as above for Formula (I) and represents hydrogen, a linear or branched alkyl group of any length, possibly substituted, or an alkenyl group of any length, possibly substituted, or an alkynyl group, possibly substituted, or a cycloalkyl group, possibly substituted, or a alkyl-substituted silyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

In another embodiment, R₁ is a hydrogen atom, an optionally substituted (C₁-C₂₀)-alkyl group, an optionally substituted (C₂-C₂₀)-alkenyl group, an optionally substituted (C₂-C₂₀)-alkynyl group, an optionally substituted (C₃-C₂₀)-cycloalkyl group, an optionally substituted (C₆-C₁₄)-aryl group, an optionally substituted (C₆-C₁₄)-heteroaryl group, an OR^{c} group or an NR^{c}₂ group, wherein the optional substituents are halogen atoms, OR^{d}, or NR^{d}₂ groups, in which R^{c} or R^{d} is a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₀)-aryl group.

In another embodiment, R₁ is a hydrogen atom, an optionally substituted (C₁-C₁₀)-alkyl group, an optionally substituted (C₂-C₁₀)-alkenyl group, an optionally substituted (C₂-C₁₀)-alkynyl group, an optionally substituted (C₃-C₁₀)-cycloalkyl group, an optionally substituted (C₆-C₁₀)-aryl group. In another embodiment, Rₜ is a hydrogen atom, an optionally substituted (C₁-C₆)-alkyl group, an optionally substituted (C₂-C₆)-alkenyl group, an optionally substituted (C₂-C₆)-alkynyl group, an optionally substituted (C₃-C₆)-cycloalkyl group, an optionally substituted (C₆)-aryl group. In another embodiment, any of the alkyl groups are fluoro-substituted, for example, a fluoro-substituted (C₁-C₆)-alkyl group, such as CF₃.

In another embodiment, the optional substituents are a (C₁-C₁₀)-alkyl group, a (C₂-C₁₀)-alkenyl group, a (C₂-C₁₀)-alkynyl group, a (C₃-C₁₀)-cycloalkyl group, or a (C₆-C₁₀)-aryl group. In another embodiment, a (C₁-C₆)-alkyl group, a (C₂-C₆)-alkenyl group, a (C₂-C₆)-alkynyl group, a (C₃-C₆)-cycloalkyl group, or a (C₆)-aryl group.

In another embodiment, R₁ is CF₃, CH₃, mesityl or tolyl.

In a general way, the compounds of Formula (III) can be prepared and isolated prior to use.

In another embodiment, R₁ in the compound of Formula (II) is as defined in all embodiments for the compound of Formula (I).

In one embodiment, R₂ represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₀)-aryl group, or a (C₆-C₁₄)-heteroaryl group, or an acyl group —O(═O)—R′, wherein R′ is a (C₁-C₂₀)-alkyl group, wherein each group is each optionally substituted with one or more halogen atoms (F, Cl, Br or I), a (C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

In one embodiment, R₂ represent a (C₁-C₁₀)-alkyl group, a (C₂-C₁₀)-alkenyl group, a (C₂-C₁₀)-alkynyl group, a (C₃-C₁₀)-cycloalkyl group, a —Si[(C₁-C₁₀)-alkyl]₃ group, a (C₆-C₁₀)-aryl group, or a (C₅-C₁₀)-heteroaryl group, or an acyl group —C(═O)—R′, wherein R′ is a (C₁-C₁₀)-alkyl group, wherein each group is each optionally substituted with one or more halogen atoms (F, Cl, Br or I), a —(C₁-C₁₀)-alkyl group, a (C₂-C₁₀)-alkenyl group, a (C₂-C₁₀)-alkynyl group, —OR^{d}, or —NR^{d}₂, wherein R^{c} and R^{d} are independently or simultaneously hydrogen, (C₁-C₁₀)-alkyl, (C₂-C₁₀)-alkenyl, or (C₂-C₁₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₁₀)-alkyl groups.

In one embodiment, R₂ represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms (F, Cl, Br or I), a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₆)-alkyl groups.

In one embodiment, R₂ represent a (C₁-C₆)-alkyl group, a —Si[(C₁-C₆)-alkyl]₃ group, or a phenyl group.

In one embodiment, R₂ represent a —Si[(C₁-C₆)-alkyl]₃ group. In one embodiment, R₂ represent a —Si[(C₁-C₃)-alkyl]₃ group. In one embodiment, R₂ represent a —Si(CH₃)₃ group.

The present disclosure also relates to cannabinol sulfonate esters of Formula (III):

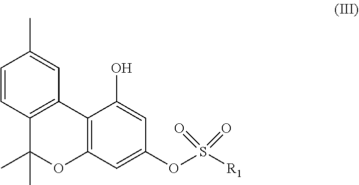

wherein,
R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group.

In a general way, the compounds of Formula (III) can be prepared and isolated prior to use.

In one embodiment, R₁ represents a hydrogen atom, —OR^{c}, —NR^{c}₂, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₀)-aryl group, or a (C₅-C₁₄)-heteroaryl group, wherein the latter 6 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In another embodiment, R₁ represents a hydrogen atom, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₀)-aryl group, a (C₅-C₁₄)-heteroaryl group, wherein the latter 6 groups are each optionally substituted with one or more halogen atoms (F, Cl, Br or I), -(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In another embodiment, R₁ represents a hydrogen atom, fluoro-substituted-(C₁-C₁₀)-alkyl, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, a (C₅-C₁₀)-heteroaryl group, wherein the latter 6 groups are each optionally substituted with one or more halogen atoms (F, Cl, Br or I), —(C₁-C₂₀)-alkyl, a (C₂-C₂₀)-alkenyl group, or a (C₂-C₂₀)-alkynyl group.

In another embodiment, R₁ represents a hydrogen atom, fluoro-substituted-(C₁-C₆)-alkyl, a (C₁-C₆)-alkyl group, a (C₂-C₆)-alkenyl group, a (C₂-C₆)-alkynyl group, a (C₃-C₆)-cycloalkyl group, a (C₆)-aryl group, a (C₆-C₆)-heteroaryl group, wherein the latter 6 groups are each optionally substituted with one or more halogen atoms (F, Cl, Br or I), or —(C₁-C₂₀)-alkyl.

In another embodiment, R₁ represents a hydrogen atom, fluoro-substituted-(C₁-C₆)-alkyl, a (C₁-C₆)-alkyl group, or a phenyl group, wherein the latter 2 groups are each optionally substituted with one or more halogen atoms (F, Cl, Br or I), or —(C₁-C₁₀)-alkyl.

In another embodiment, R₁ represents a hydrogen atom, —CF₃,

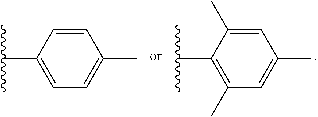

In one embodiment, the compound of Formula (III) is one of the structures below:

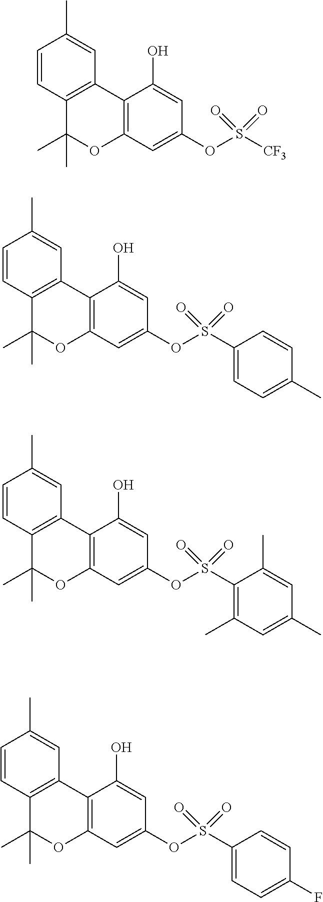

The present disclosure relates to a cannabinol precursor of Formula (IV):

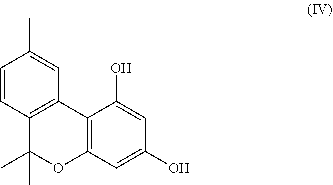 [SMILES: Cc1ccc2c(c1)-c1c(O)cc(O)cc1OC2(C)C]

The present disclosure also relates to a cannabinol precursor of Formula (IV), wherein one or more of the hydrogen atoms are replaced with deuterium.

The present disclosure also relates to a cannabinol precursor of Formula (IV), wherein one or more of the carbon-12 atoms are replaced with carbon-13.

In another embodiment, the present disclosure relates to cannabinol precursors of Formula (V):

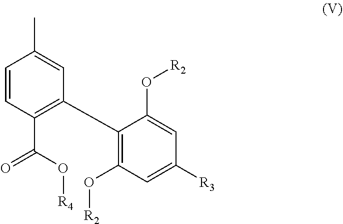

wherein,
one or more of the hydrogen atoms are replaced with deuterium and/or one or more of the carbon-12 atoms are replaced with carbon-13;
R₂ is as defined in any of the embodiments above and represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ and R₄ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₃ and R₄ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₆-C₁₄)-heteroaryl group, wherein the latter 6 groups are each optionally substituted with one or more halogen atoms (F, Cl, Br or I), or —(C₁-C₂₀)-alkyl.

In one embodiment, R₃ and R₄ represents a hydrogen atom, a (C₁-C₁₀)-alkyl group, a (C₂-C₁₀)-alkenyl group, a (C₂-C₁₀)-alkynyl group, a (C₃-C₁₀)-cycloalkyl group, a (C₆-C₁₀)-aryl group, or a (C₅-C₁₀)-heteroaryl group, wherein the latter 6 groups are each optionally substituted with one or more halogen atoms (F, Cl, Br or I), or —(C₁-C₁₀)-alkyl.

In one embodiment, R₃ and R₄ represents a hydrogen atom, a (C₁-C₆)-alkyl group, a (C₂-C₆)-alkenyl group, a (C₂-C₆)-alkynyl group, a (C₃-C₆)-cycloalkyl group, a (C₆)-aryl group, or a (C₆-C₆)-heteroaryl group, wherein the latter 6 groups are each optionally substituted with one or more halogen atoms (F, Cl, Br or I), or —(C₁-C₆)-alkyl.

In another embodiment, the present disclosure relates to cannabinol precursors of Formula (VI):

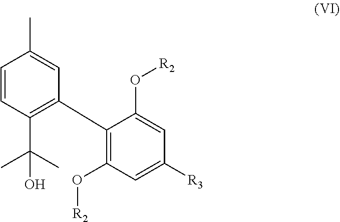

wherein,
one or more of the hydrogen atoms are replaced with deuterium and/or one or more of the carbon-12 atoms are replaced with carbon-13;
R₂ is as defined above in any of the embodiments and represents a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ is as defined above in any embodiment and represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In another embodiment, the present disclosure relates to cannabinol compounds of Formula (VII):

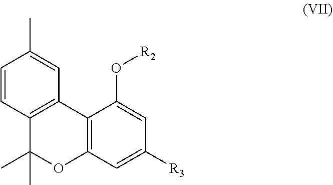

wherein,
one or more of the hydrogen atoms in the p-cymene fragment of the molecule are replaced with deuterium and/or one or more of the carbon-12 atoms in the p-cymene fragment of the molecule are replaced with carbon-13;
R₂ is as defined above in any embodiment and represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ is as defined above in any embodiment and represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

The transformations to which the compounds of the disclosure can be applied include but are not limited to catalytic and non-catalytic carbon-carbon bond forming reactions including Ullman, Suzuki-Miyaura, Negishi, Kumada, Sonogashira and Stille reactions. Such carbon-carbon bond forming reactions include the use of compounds of the disclosure to prepare cannabinol compounds of Formula (VII):

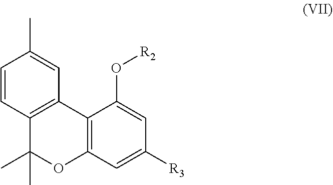

### and Formula (VIII):

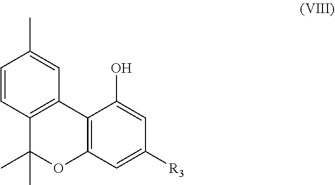

wherein,
R₂ is as defined above in any embodiment and represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ is as defined in any embodiment above and represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₂ in the compounds of Formula (VII) and (VIII) are as defined in each embodiment for the compounds of Formula (I) to (VI).

In one embodiment, R₃ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₀)-aryl group, wherein the latter 5 groups are each optionally substituted with one or more halogen atoms (F, Cl, Br or I), -(C₁-C₂₀)-alkyl, a (C₂-C₂₀)-alkenyl group, a (C₂-C₂₀)-alkynyl group, (C₆-C₁₀)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In one embodiment, R₃ represents a hydrogen atom, a (C₁-C₂₀)-alkyl group, a (C₂-C₂₀)-alkenyl group, a (C₆-C₁₀)-aryl group, wherein the latter 3 groups are each optionally substituted with one or more halogen atoms (F, Cl, Br or I), —(C₁-C₁₀)-alkyl, a (C₂-C₁₀)-alkenyl group, a (C₂-C₁₀)-alkynyl group, or (C₆-C₁₀)-aryl group.

In one embodiment, R₃ represents a hydrogen atom, a (C₁-C₂₀)-alkyl group, a (C₆-C₁₀)-aryl group, wherein the latter 2 groups are each optionally substituted with one or more phenyl groups.

In one embodiment, R₃ represents a hydrogen atom or a (C₁-C₂₀)-alkyl group optionally substituted with a phenyl group.

In some other aspects, the present disclosure also relates to the preparation of cannabinol compounds of Formula (VII) and Formula (VIII), wherein one or more of the hydrogen atoms are replaced with deuterium.

In some other aspects, the present disclosure also relates to the preparation of cannabinol compounds of Formula (VII) and Formula (VIII), wherein one or more of the carbon-12 atoms are replaced with carbon-13 atoms.

In some other aspects, the present disclosure also relates to the preparation of cannabinol compounds of Formula (VII) and Formula (VIII), wherein one or more of the hydrogen atoms of the p-cymene fragment of the molecule are replaced with deuterium.

In some other aspects, the present disclosure also relates to the preparation of cannabinol compounds of Formula (VII) and Formula (VIII), wherein one or more of the carbon-12 atoms of the p-cymene fragment of the molecule are replaced with carbon-13 atoms.

### (III) Processes of the Disclosure

The present disclosure also relates to a process for the production of compounds of Formula (IV) comprising first contacting a compound of Formula (IX)

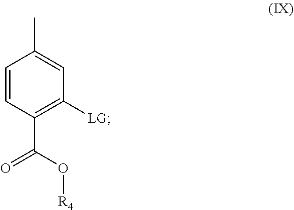

and a compound of Formula (X),

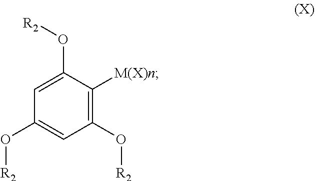

In the presence of a catalyst to form a compound of Formula (XI).

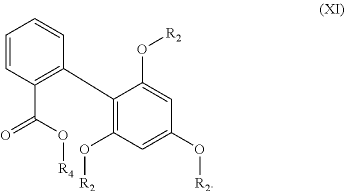

Compound (XI) is then transformed to a compound of Formula (IV) by contacting a compound of Formula (XI) with methylmagnesium Grignard, followed by removal of the R₂ groups and ring closure.

Compound (IV) is then transformed to a compound of Formula (III) by contacting a compound of Formula (IV) with a sulphonating agent in the presence of a base.

Compound (III) is then transformed to a compound of Formula (II) by contacting a compound of Formula (III) with a suitable reagent in the presence of a base.

In some aspects, the transformation of Compound (IX) and Compound (X) to Compound (XI) requires a suitable catalyst. Suitable catalysts include but are not limited to transition metal salts and complexes, such as compounds of palladium, nickel, iron, ruthenium, cobalt, rhodium, iridium and copper.

The disclosure also relates to a process for the catalytic and non-catalytic use of compounds of Formula (I), Formula (II) and Formula (III) to prepare cannabinol compounds of Formula (VII) and Formula (VIII):

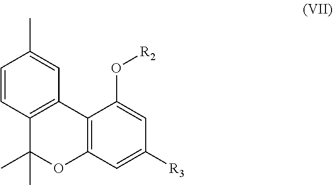

### and Formula (VIII):

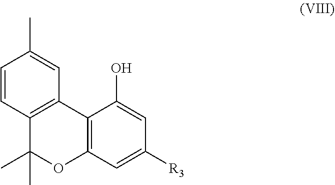

wherein,
R₂ represents hydrogen, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups; and
R₃ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted.

In one embodiment, R₂ in the compounds of Formula (VII) and (VIII) are as defined in each embodiment for the compounds of Formula (I) to (VI).

In one embodiment, R₃ represents a hydrogen atom, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₀)-aryl group, wherein the latter 5 groups are each optionally substituted with one or more halogen atoms (F, Cl, Br or I), -(C₁-C₂₀)-alkyl, a (C₂-C₂₀)-alkenyl group, a (C₂-C₂₀)-alkynyl group, (C₆-C₁₀)-aryl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

In one embodiment, R₃ represents a hydrogen atom, a (C₁-C₂₀)-alkyl group, a (C₂-C₂₀)-alkenyl group, a (C₆-C₁₀)-aryl group, wherein the latter 3 groups are each optionally substituted with one or more halogen atoms (F, Cl, Br or I), -(C₁-C₁₀)-alkyl, a (C₂-C₁₀)-alkenyl group, a (C₂-C₁₀)-alkynyl group, or (C₆-C₁₀)-aryl group.

In one embodiment, R₃ represents a hydrogen atom, a (C₁-C₂₀)-alkyl group, a (C₆-C₁₀)-aryl group, wherein the latter 2 groups are each optionally substituted with one or more phenyl groups.

In one embodiment, R₃ represents a hydrogen atom or a (C₁-C₂₀)-alkyl group optionally substituted with a phenyl group.

In some other aspects, the present disclosure also relates to the preparation of cannabinol compounds of Formula (VII) and Formula (VIII), wherein one or more of the hydrogen atoms are replaced with deuterium.

In some other aspects, the present disclosure also relates to the preparation of cannabinol compounds of Formula (VII) and Formula (VIII), wherein one or more of the carbon-12 atoms are replaced with carbon-13 atoms.

In some other aspects, the present disclosure also relates to the preparation of cannabinol compounds of Formula (VII) and Formula (VIII), wherein one or more of the hydrogen atoms of the p-cymene fragment of the molecule are replaced with deuterium.

Carbon-carbon bond forming reactions for the preparation of cannabinol compounds of Formula (VII) and Formula (VIII) include but are not limited to catalytic and non-catalytic Ullman, Suzuki-Miyaura, Negishi, Kumada, Sonogashira and Stille reactions.

In some embodiments of the disclosure, a compound of Formula (I), Formula (II) or Formula (III) is contacted with a nucleophilic R₃ group, R₃—W wherein R₃ is as defined above and is nucleophilic and W is an electrophilic group, such as a boron containing compound such as R₃—B(OH)₂, R₃—B(OR)₂ or R₃—BF₃K; or a Grignard compound such as R₃—MgX; or an organozinc compound, such as R₃—ZnX, in the presence or absence of a catalyst to produce a compound of Formula (VII) or Formula (VIII).

In one embodiment, compounds of Formula (I) are prepared as in the following examples:

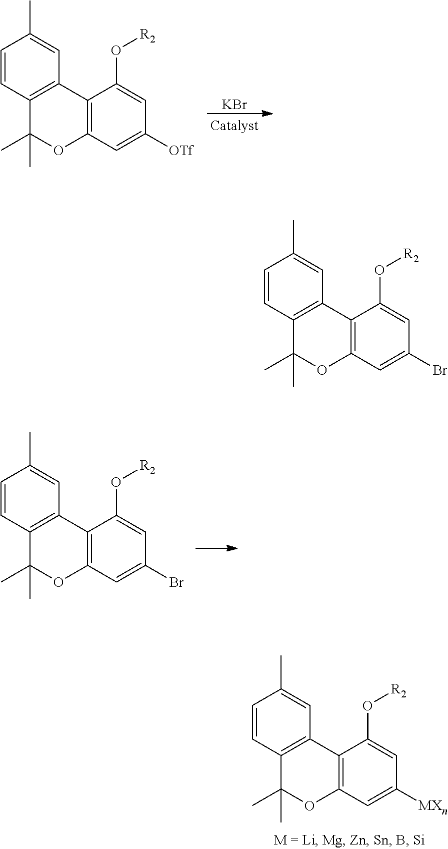

The present disclosure also relates to a process for the production of compounds of Formula (V) comprising first contacting a compound of Formula (IX)

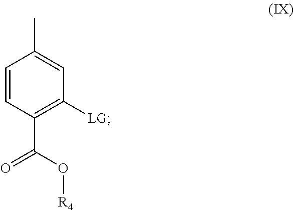

and a compound of Formula (XII),

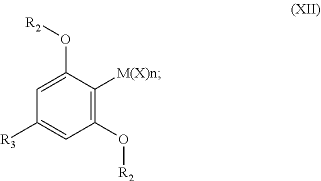

in the presence of a catalyst to form a compound of Formula (V)

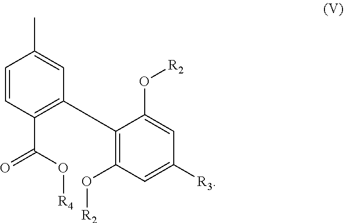

Compound (V) is then transformed to a Compound of Formula (VI):

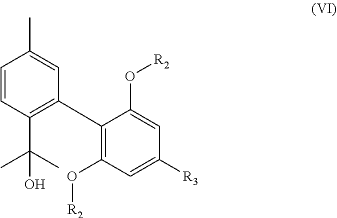

by contacting a compound of Formula (V) with methylmagnesium Grignard.

Compound (VI) is converted to a cannabinol compound by removal of the R₂ groups and ring closure.

In some embodiments of the disclosure, the catalytic system characterizing the process of the instant disclosure may comprise a base. In some embodiments, said base can be any conventional base. In some embodiments, non-limiting examples include: organic non-coordinating bases such as DBU, an alkaline or alkaline-earth metal carbonate, a carboxylate salt such as sodium or potassium acetate, or an alcoholate or hydroxide salt. Preferred bases are the alcoholate or hydroxide salts selected from the group consisting of the compounds of formula (RO)₂M′ and ROM″, wherein M′ is an alkaline-earth metal, M″ is an alkaline metal and R stands for hydrogen or a linear or branched alkyl group.

The catalyst can be added to the reaction medium in a large range of concentrations. As non-limiting examples, one can cite as catalyst concentration values ranging from 0.001% to 50%, relative to the amount of substrate, thus representing respectively a substrate/catalyst (S/cat) ratio of 100,000 to 2. Preferably, the complex concentration will be comprised between 0.01% and 10%, i.e. a S/cat ratio of 10,000 to 10 respectively. In some preferred embodiments, there will be used concentrations in the range of 0.1 to 5%, corresponding to a S/cat ratio of 1000 to 20 respectively.

If required, useful quantities of base, added to the reaction mixture, may be comprised in a relatively large range. In some embodiments, non-limiting examples include: ranges between 1 to 100 molar equivalents relative to the substrate. However, it should be noted that it is also possible to add a small amount of base (e.g. base/substrate=1 to 3) to achieve high yields.

In the processes of this disclosure, the catalytic reaction can be carried out in the presence or absence of a solvent. When a solvent is required or used for practical reasons, then any solvent currently used in catalytic reactions can be used for the purposes of the disclosure. Non-limiting examples include aromatic solvents such as benzene, toluene or xylene, hydrocarbon solvents such as hexane or cyclohexane, ethers such as tetrahydrofuran, or yet primary or secondary alcohols, or water, or mixtures thereof. A person skilled in the art is well able to select the solvent most convenient in each case to optimize the catalytic reaction.

The temperature at which the catalytic reaction can be carried out is comprised between −30° C. and 200° C., more preferably in the range of between 0° C. and 100° C. Of course, a person skilled in the art is also able to select the preferred temperature.

Standard catalytic conditions, as used herein, typically implies the mixture of the substrate with the catalyst with or without a base, possibly in the presence of a solvent, and then treating such a mixture with the desired reactant at a chosen temperature in air or under an inert atmosphere of nitrogen or argon gas. Varying the reaction conditions, including for example, catalyst, temperature, solvent and reagent, to optimize the yield of the desired product would be well within the abilities of a person skilled in the art.

The present disclosure is described in the following Examples, which are set forth to aid in the understanding of the disclosure, and should not be construed to limit in any way the scope of the disclosure as defined in the claims which follow thereafter.

### (IV) Benzyl Cannabinols

The present disclosure also includes benzyl cannabinols having the following structure:

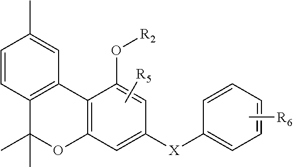

wherein
R₂ is as defined above in any paragraph for compounds of the Formula (III) to Formula (VII);
R₅ and R₆ are one or more substituents which are hydrogen, halo, —OR^{c}, —NR^{c}₂, carboxylates (—COOR, where R is H or (C₁-C₆)-alkyl), phosphates, sulfates, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₀)-aryl group, or a (C₅-C₁₄)-heteroaryl group, wherein R^{c} and R^{d} are independently or simultaneously hydrogen, (C₁-C₂₀)-alkyl, (C₂-C₂₀)-alkenyl, or (C₂-C₂₀)-alkynyl,
X is (C₁-C₁₀)-alkylene) or (C₂-C₁₀)-alkenylene), and all isomers, and salts thereof.

In one embodiment, R₅ and R₆ are one or more substitutents which are hydrogen, halo, a (C₁-C₁₀)-alkyl group, or a (C₅-C₁₀)-aryl group. In one embodiment, R₅ and R₆ are one or more substituents which are hydrogen, halo, a (C₁-C₆)-alkyl group, or a phenyl group.

In one embodiment, X is (C₁-C₆)-alkylene) or (C₂-C₆)-alkenylene). In another embodiment, X is (C₁-C₂)-alkylene) or (C₂)-alkenylene).

In one embodiment, the compound of the Formula (X) is one of the compounds below:

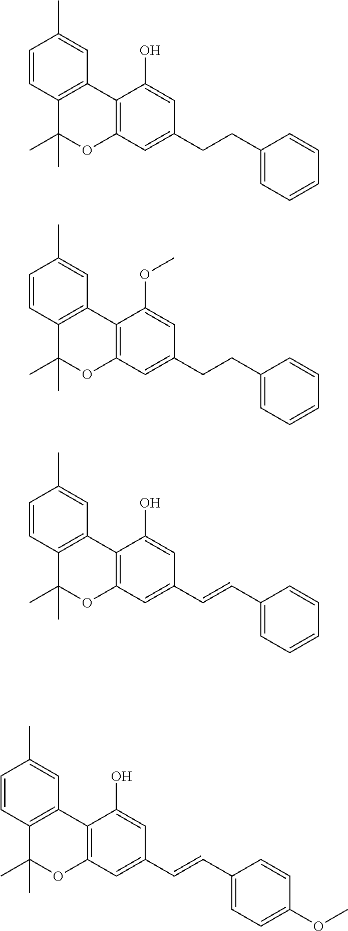 [SMILES: Cc1ccc2c(c1)-c1c(O)cc(CCc3ccccc3)cc1OC2(C)C ; COc1cc(CCc2ccccc2)cc2c1-c1cc(C)ccc1C(C)(C)O2 ; Cc1ccc2c(c1)-c1c(O)cc(/C=C/c3ccccc3)cc1OC2(C)C ; COc1ccc(/C=C/c2cc(O)c3c(c2)OC(C)(C)c2ccc(C)cc2-3)cc1]

### EXAMPLES

The disclosure will now be described in further details by way of the following examples, wherein the temperatures are indicated in degrees centigrade and the abbreviations have the usual meaning in the art.

All the procedures described hereafter have been carried out under an inert atmosphere unless stated otherwise. All preparations and manipulations under air-free conditions were carried out under N₂ or Ar atmospheres with the use of standard Schlenk, vacuum line and glove box techniques in dry, oxygen-free solvents. Deuterated solvents were degassed and dried over activated molecular sieves. NMR spectra were recorded on a 400 MHz spectrometer (400 MHz for ¹H, 100 MHz for ¹³C, 376 MHz for ¹⁹F and 162 MHz for ³¹P). All ³¹P chemical shifts were measured relative to 85% H3PO4 as an external reference. ¹H and ¹³C chemical shifts were measured relative to partially deuterated solvent peaks but are reported relative to tetramethylsilane.

### Example 1

### Preparation of methyl 2′,4′,6′-trimethoxy-5-methylbiphenyl-2-carboxylate

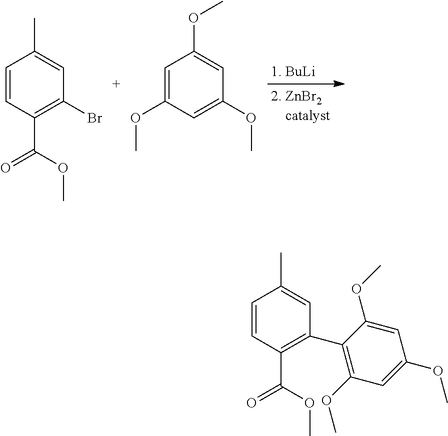

Anhydrous THF (12 ml) was added to 1,3,5-trimethoxybenzene (5.35 g, 32 mmol) under argon. A solution of n-butyllithium (20 ml, 1.6 M in hexanes, 32 mmol) was added and the mixture was refluxed at 40° C. for 2 hours. The solvent was removed under reduced pressure and anhydrous THF (20 ml) was added and the resulting yellow solution was cooled to 0° C. Solid ZnBr₂ (7.22 g, 32 mmol) was added under a flow of argon. The mixture heated to boiling spontaneously. Stirring was continued for 30 minutes. The catalyst PdCl₂(dppf) (0.35 g, 0.48 mmol) was added followed by methyl 2-bromo-4-methylbenzoate 7.10 g, 31 mmol) and the mixture heated at 70° C. for 72 hours. The reaction was quenched with water, followed by ammonium chloride solution. The aqueous layer was extracted with CH₂Cl₂ (3×50 ml) and the combined organic fraction was dried (MgSO₄), filtered and evaporated to dryness. Purification by column chromatography (EA/hexanes, 1:4) gave the product as a crystalline white solid. Yield=7.20 g.

### Example 2

### Preparation of 2-(2′,4′,6′-trimethoxy-5-methylbiphenyl-2-yl)propan-2-ol

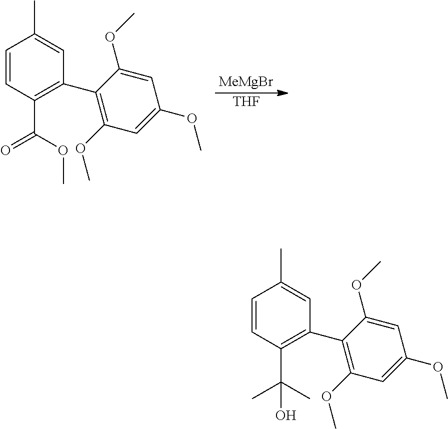 [SMILES: "MeMgBr" ; COC(=O)c1ccc(C)cc1-c1c(OC)cc(OC)cc1OC ; COc1cc(OC)c(-c2cc(C)ccc2C(C)(C)O)c(OC)c1 ; "THF"]

A solution of MeMgBr (35 ml, 3 M solution in ether, 105 mmol) was added to a solution of methyl 2′,4′,6′-trimethoxy-5-methylbiphenyl-2-carboxylate (7.2 g, 23 mmol) in THF (60 ml) under argon and the mixture stirred for 2 hours at room temperature. The reaction was quenched with water, followed by ammonium chloride solution. The aqueous layer was extracted with CH₂Cl₂ (3×50 ml) and the combined organic fraction was dried (MgSO₄), filtered and evaporated to dryness. Yield=7.18 g.

### Example 3

### Preparation of 6,6,9-trimethyl-6H-benzo[c]chromene-1,3-diol

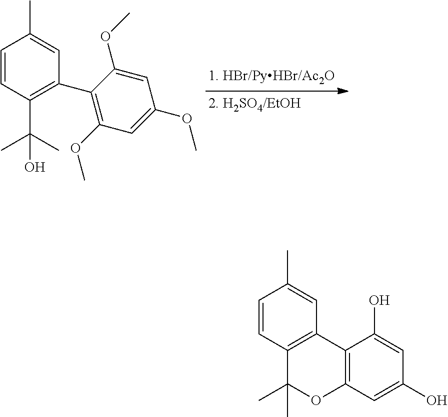 [SMILES: COc1cc(OC)c(-c2cc(C)ccc2C(C)(C)O)c(OC)c1 ; Cc1ccc2c(c1)-c1c(O)cc(O)cc1OC2(C)C]

Pyridinium bromide (25.5 g, 159 mmol), hydrobromic acid (48%, 13.4 g, 79.5 mmol) and acetic anhydride (30 ml) were added to 2-(2′,4′,6′-trimethoxy-5-methylbiphenyl-2-yl)propan-2-ol (5.0 g, 15.9 mmol) and the mixture was heated at 115° C. for 18 hours. It was cooled to room temperature and diluted with water. It was neutralized with NaOH solution to about pH 6, then extracted with CH₂Cl₂ (3×50 ml). The organic fraction was dried (MgSO₄), then filtered, and evaporated to dryness. The residue was suspended in a mixture of H₂SO₄ (2M, 25 ml) and ethanol (25 ml) and stirred for 18 hours. It was neutralized with NaOH solution to about pH 6, then extracted with CH₂Cl₂ (3×50 ml). The organic fraction was dried (MgSO₄), filtered, and evaporated to dryness. Recrystallization from hexanes gave the product as a white, crystalline solid. Yield=3.30 g.

### Example 4

### Preparation of 1-hydroxy-6,6,9-trimethyl-6H-benzo[c]chromen-3-yl trifluoromethanesulfonate

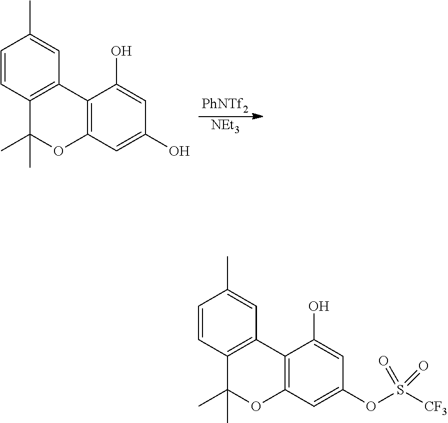 [SMILES: "PhNTf2" ; Cc1ccc2c(c1)-c1c(O)cc(O)cc1OC2(C)C ; Cc1ccc2c(c1)-c1c(O)cc(OS(=O)(=O)C(F)(F)F)cc1OC2(C)C ; "NEt3"]

Triethylamine (5.1 g, 50.4 mmol) was added to a solution of 6,6,9-trimethyl-6H-benzo[c]chromene-1,3-diol (4.31 g, 16.8 mmol) in dichloromethane (50 ml) and the mixture cooled to 0° C. Solid N-Phenyl-bis(trifluoromethanesulfonimide) (6.30 g, 17.7 mmol) was added over 30 minutes and the mixture was stirred at room temperature overnight. It was quenched with water (50 ml) and the phases separated. The aqueous layer was extracted with dichloromethane (3×25 ml) and the combined organic layers were dried (MgSO₄). It was filtered through a short pad of silica gel and the solvent was removed under reduced pressure. Chromatography using hexanes/EA (15/1) gave the product as a crystalline white solid. Yield=4.32 g.

### Example 5

### Preparation of 6,6,9-trimethyl-1-(trimethylsilyloxy)-6H-benzo[c]chromen-3-yl trifluoromethanesulfonate

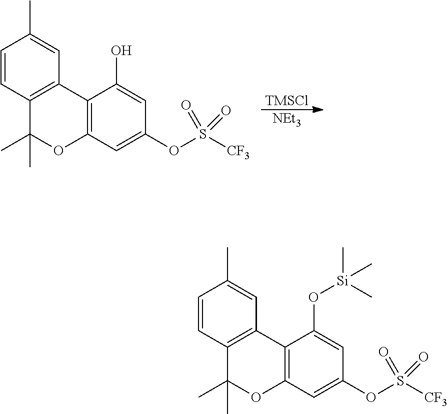 [SMILES: "TMSCl" ; Cc1ccc2c(c1)-c1c(O)cc(OS(=O)(=O)C(F)(F)F)cc1OC2(C)C ; Cc1ccc2c(c1)-c1c(cc(OS(=O)(=O)C(F)(F)F)cc1O[Si](C)(C)C)OC2(C)C ; "NEt3"]

TMSCl (1.45 g, 13.4 mmol) was added to a mixture of 1-hydroxy-6,6,9-trimethyl-6H-benzo[c]chromen-3-yl trifluoromethanesulfonate (2.6 g, 6.67 mmol) and NEt₃ (1.35 g, 13.4 mmol) in CH₂Cl₂ (25 ml) at room temperature. The mixture was stirred overnight, then filtered and the solids were washed with dichloromethane. The volatiles were removed from the combined filtrate under reduced pressure. The residue was suspended in hexanes (25 ml) and stirred for 2 hours at room temperature. The mixture was filtered, and the solvent was removed. The residue was dried under vacuum to give the product as a viscous yellow oil. Yield=2.98 g.

### Example 6

### Preparation of 6,6,9-trimethyl-3-pentyl-6H-benzo[c]chromen-1-ol (cannabinol, CBN)

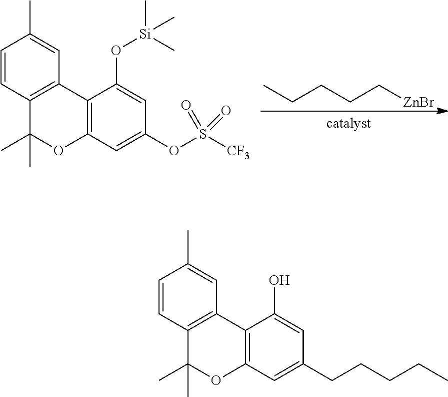

A solution of n-pentylzinc bromide (5.56 ml of a 0.5 M solution in THF, 2.82 mmol) was added to a mixture of 6,6,9-trimethyl-1-(trimethylsilyloxy)-6H-benzo[c]chromen-3-yl trifluoromethanesulfonate (1.0 g, 2.17 mmol) and PdCl₂(dppf) (40 mg, 0.054 mmol) in THF (4 ml) and the mixture was stirred at 45° C. for 18 hours under argon. It was cooled to room temperature and water (10 ml) was added followed by 2M H₂SO₄ (10 ml) and the mixture stirred at room temperature for 1 hour. The phases were separated, and the aqueous layer was extracted with ether (3×10 ml). The combined organic layer was dried (MgSO₄), filtered and evaporated to dryness. The residue was dissolved in hexanes and filtered through a short pad of silica gel. The silica was washed with hexanes and the combined filtrate was evaporated to dryness to give the product as a pale-yellow oil. Yield=0.65 g.

### Example 7

### Preparation of 6,6,9-trimethyl-3-propyl-6H-benzo[c]chromen-1-ol (cannabivarin, CBNV)

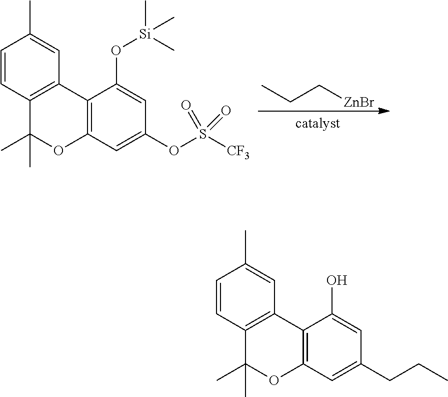

This was prepared using the procedure described in Example 6, using n-propylzinc bromide. The product was isolated as a pale-yellow oil. Yield=0.58 g.

### Example 8

### Preparation of 3-heptyl-6,6,9-trimethyl-6H-benzo[c]chromen-1-ol (Cannabiphorol, CBNP)

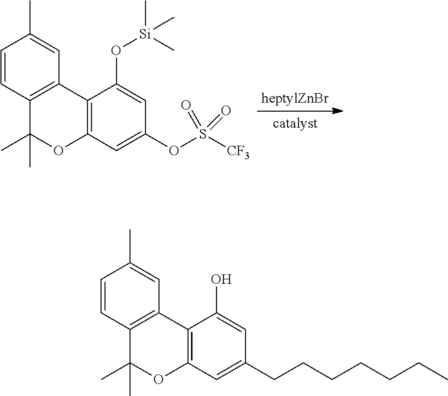 [SMILES: Cc1ccc2c(c1)-c1c(cc(OS(=O)(=O)C(F)(F)F)cc1O[Si](C)(C)C)OC2(C)C ; CCCCCCCc1cc(O)c2c(c1)OC(C)(C)c1ccc(C)cc1-2]

This was prepared using the procedure described in Example 6, using n-heptylzinc bromide. The product was isolated as a pale-yellow oil. Yield=0.72 g.

### Example 9. Preparation of 6,6,9-trimethyl-3-phenethyl-6H-benzo[c]chromen-1-ol

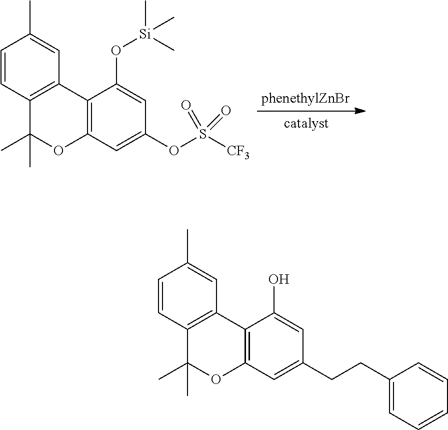 [SMILES: Cc1ccc2c(c1)-c1c(cc(OS(=O)(=O)C(F)(F)F)cc1O[Si](C)(C)C)OC2(C)C ; Cc1ccc2c(c1)-c1c(O)cc(CCc3ccccc3)cc1OC2(C)C]

This was prepared using the procedure described in Example 6, using phenethylzinc bromide. The product was isolated as a pale-yellow oil. Yield=0.74 g.

### Example 10

### Preparation of 3-butyl-6,6,9-trimethyl-6H-benzo[c]chromen-1-ol (Cannabibutol, CBNB)

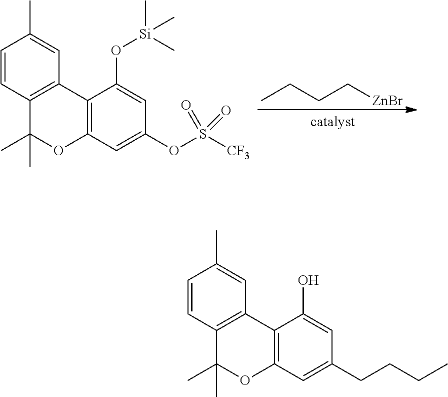

This was prepared using the procedure described in Example 6, using butylzinc bromide. The product was isolated as a pale-yellow oil. Yield=0.62 g.

### Example 11

### Preparation of 3-hexyl-6,6,9-trimethyl-6H-benzo[c]chromen-1-ol (Cannabihexol, CBNH)

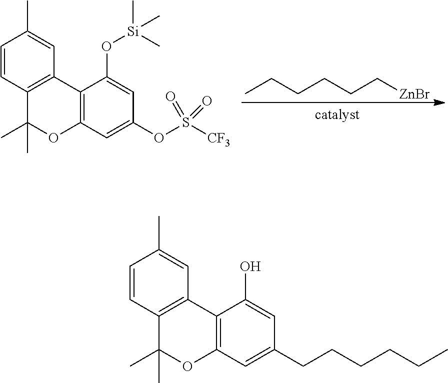

This was prepared using the procedure described in Example 6, using hexylzinc bromide. The product was isolated as a pale-yellow oil. Yield=0.67 g.

### Example 12

### Preparation of 3-ethyl-6,6,9-trimethyl-6H-benzo[c]chromen-1-ol

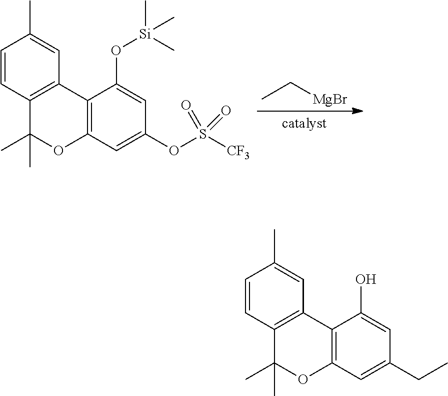

A solution of ethylmagnesium bromide (5.2 ml of a 1.0 M solution in THF, 5.2 mmol) was added to zinc bromide (1.17 g, 5.21 mmol) and the resulting solution was added under argon to a mixture of 6,6,9-trimethyl-1-(trimethylsilyloxy)-6H-benzo[c]chromen-3-yl trifluoromethanesulfonate (0.8 g, 1.74 mmol) and PdCl₂(dppf) (32 mg, 0.043 mmol) in THF (4 ml) and the mixture was stirred at 45° C. for 18 hours under argon. It was cooled to room temperature and water (10 ml) was added followed by 2M H₂SO₄ (10 ml) and the mixture stirred at room temperature for 1 hour. The phases were separated, and the aqueous layer was extracted with ether (3×10 ml). The combined organic layer was dried (MgSO₄), filtered and evaporated to dryness. The residue was dissolved in hexanes and filtered through a short pad of silica gel. The silica was washed with hexanes and the combined filtrate was evaporated to dryness to give the product as a pale-yellow oil. Yield=0.38 g.

### Example 13

### Preparation of 3,6,6,9-tetramethyl-6H-benzo[c]chromen-1-ol

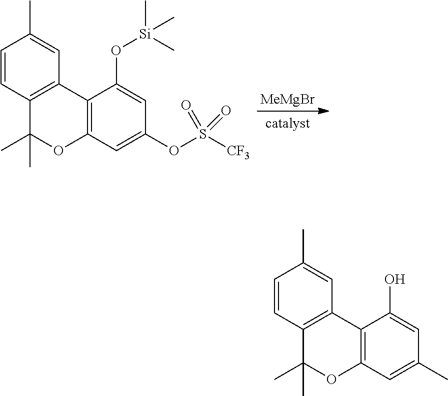 [SMILES: "MeMgBr" ; Cc1ccc2c(c1)-c1c(cc(OS(=O)(=O)C(F)(F)F)cc1O[Si](C)(C)C)OC2(C)C ; Cc1cc(O)c2c(c1)OC(C)(C)c1ccc(C)cc1-2]

This was prepared using the procedure described in Example 12, using methylmagnesium bromide. The product was isolated as a pale-yellow oil. Yield=0.37 g.

### Example 14

### Preparation of 2-(2′,4′,6′-trimethoxy-5-methyl-[1,1′-biphenyl]-2-yl)propan-1,1,1,3,3,3-d₆-2-ol

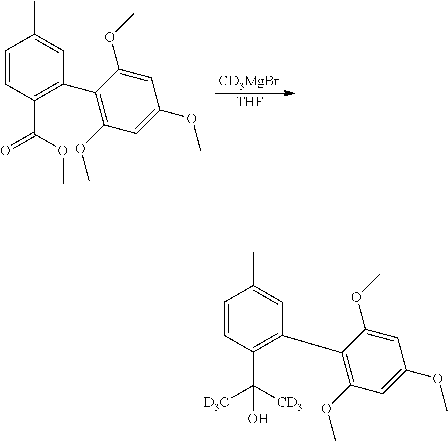 [SMILES: COC(=O)c1ccc(C)cc1-c1c(OC)cc(OC)cc1OC ; "CD3MgBr" ; [2H]C([2H])([2H])C(O)(c1ccc(C)cc1-c1c(OC)cc(OC)cc1OC)C([2H])([2H])[2H] ; "THF"]

This was prepared using the procedure described in Example 2, using CD₃MgBr.

### Example 15

### Preparation of 9-methyl-6,6-bis(methyl-d₃)-6H-benzo[c]chromene-1,3-diol

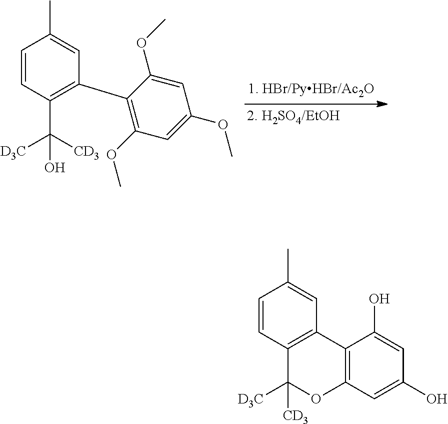 [SMILES: [2H]C([2H])([2H])C(O)(c1ccc(C)cc1-c1c(OC)cc(OC)cc1OC)C([2H])([2H])[2H] ; [2H]C([2H])([2H])C1(C([2H])([2H])[2H])Oc2cc(O)cc(O)c2-c2cc(C)ccc21]

This was prepared using the procedure described in Example 3.

### Example 16

### Preparation of 1-hydroxy-9-methyl-6,6-bis(methyl-d₃)-6H-benzo[c]chromen-3-yl trifluoromethanesulfonate

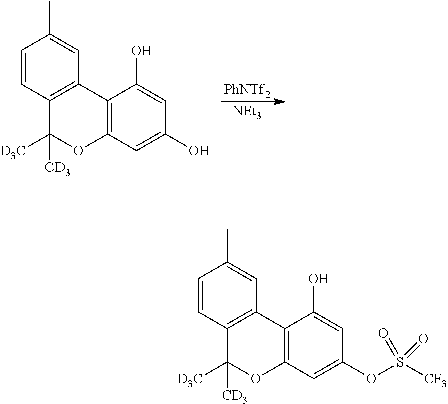 [SMILES: "PhNTf2" ; [2H]C([2H])([2H])C1(C([2H])([2H])[2H])Oc2cc(O)cc(O)c2-c2cc(C)ccc21 ; [2H]C([2H])([2H])C1(C([2H])([2H])[2H])Oc2cc(OS(=O)(=O)C(F)(F)F)cc(O)c2-c2cc(C)ccc21 ; "NEt3"]

This was prepared using the procedure described in Example 4.

### Example 17

### Preparation of 9-methyl-6,6-bis(methyl-d₃)-1-((trimethylsilyl)oxy)-6H-benzo[c]chromen-3-yl trifluoromethanesulfonate

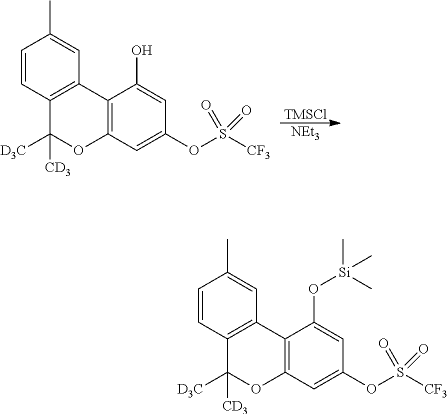 [SMILES: "TMSCl" ; [2H]C([2H])([2H])C1(C([2H])([2H])[2H])Oc2cc(OS(=O)(=O)C(F)(F)F)cc(O)c2-c2cc(C)ccc21 ; [2H]C([2H])([2H])C1(C([2H])([2H])[2H])Oc2cc(OS(=O)(=O)C(F)(F)F)cc(O[Si](C)(C)C)c2-c2cc(C)ccc21 ; "NEt3"]

This was prepared using the procedure described in Example 5.

### Example 18

### Preparation of 9-methyl-6,6-bis(methyl-d₃)-3-pentyl-6H-benzo[c]chromen-1-ol

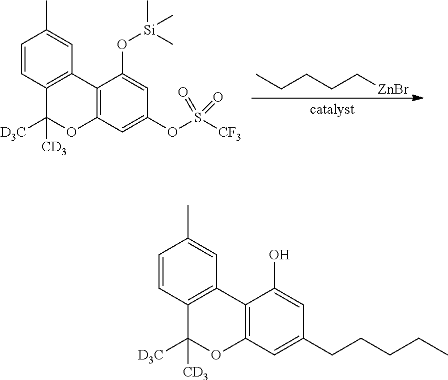

This was prepared using the procedure described in Example 6.

### Example 19

### Preparation of 2-(2′,4′,6′-trimethoxy-5-methyl-[1,1′-biphenyl]-2-yl)propan-2-ol-1,3-¹³C₂

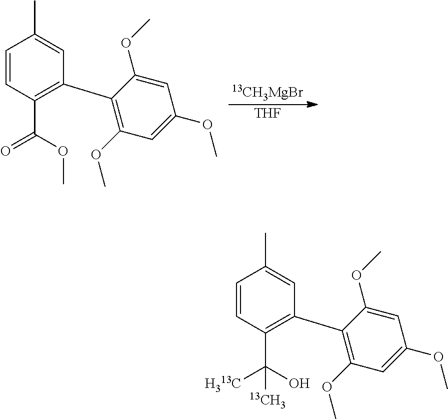

This was prepared using the procedure described in Example 2, using ¹³CH₃MgBr.

### Example 20

### Preparation of 9-methyl-6,6-di(methyl-¹³C)-6H-benzo[c]chromene-1,3-diol

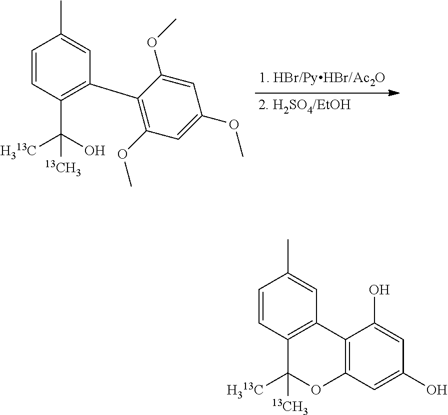 [SMILES: COc1cc(OC)c(-c2cc(C)ccc2C([13CH3])([13CH3])O)c(OC)c1 ; Cc1ccc2c(c1)-c1c(O)cc(O)cc1OC2([13CH3])[13CH3]]

This was prepared using the procedure described in Example 3.

### Example 21

### Preparation of 1-hydroxy-9-methyl-6,6-di(methyl-¹³C)-6H-benzo[c]chromen-3-yl trifluoromethanesulfonate

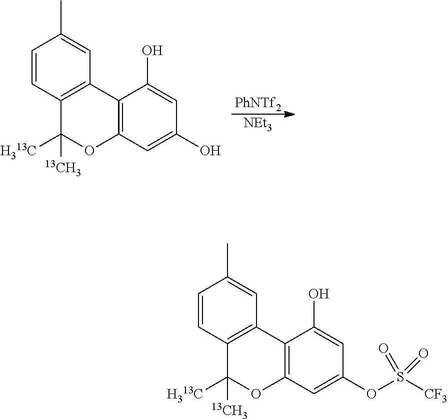 [SMILES: "PhNTf2" ; Cc1ccc2c(c1)-c1c(O)cc(O)cc1OC2([13CH3])[13CH3] ; Cc1ccc2c(c1)-c1c(O)cc(OS(=O)(=O)C(F)(F)F)cc1OC2([13CH3])[13CH3] ; "NEt3"]

This was prepared using the procedure described in Example 4.

### Example 22

### Preparation of 9-methyl-6,6-dimethyl-¹³C)-1-((trimethylsilyl)oxy)-6H-benzo[c]chromen-3-yl trifluoromethanesulfonate

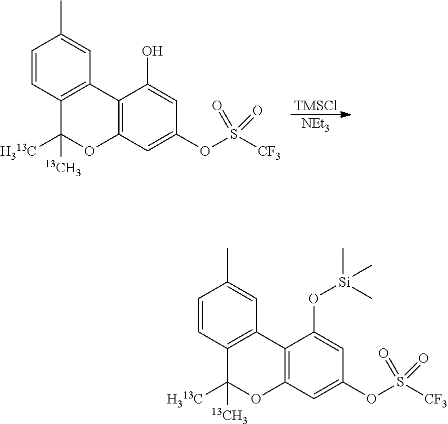 [SMILES: "TMSCl" ; Cc1ccc2c(c1)-c1c(O)cc(OS(=O)(=O)C(F)(F)F)cc1OC2([13CH3])[13CH3] ; Cc1ccc2c(c1)-c1c(cc(OS(=O)(=O)C(F)(F)F)cc1O[Si](C)(C)C)OC2([13CH3])[13CH3] ; "NEt3"]

This was prepared using the procedure described in Example 5.

### Example 23

### Preparation of 9-methyl-6,6-di(methyl-¹³C)-3-pentyl-6H-benzo[c]chromen-1-ol

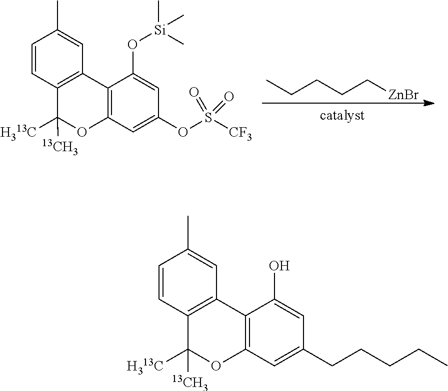

This was prepared using the procedure described in Example 6.

### Example 24

### Preparation of 1-methoxy-6,6,9-trimethyl-6H-benzo[c]chromen-3-yl trifluoromethanesulfonate

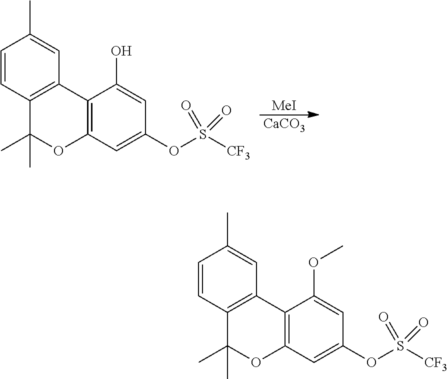

Anhydrous acetonitrile (10 ml) was added to a mixture of 1-hydroxy-6,6,9-trimethyl-6H-benzo[c]chromen-3-yl trifluoromethanesulfonate (1.0 g, 2.57 mmol), methyl iodide (0.44 g, 3.1 mmol), and potassium carbonate (0.31 g, 3.1 mmol) and the suspension stirred vigorously under argon for 12 hours at room temperature. The mixture was evaporated to dryness and the residue was extracted with ether. The ether extract was washed with water, and dried (MgSO₄). It was filtered and the solvent removed under reduced pressure. The residue was dried under vacuum to give the product as a pale-yellow oil. Yield=1.02 g.

### Example 25

### Preparation of methyl methyl 2′,6′-dimethoxy-5-methyl-4′-pentylbiphenyl-2-carboxylate

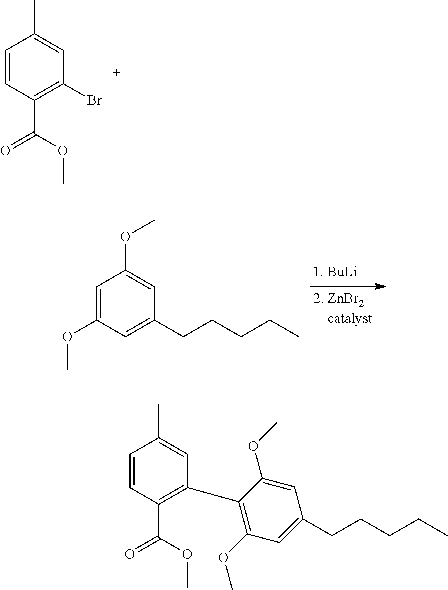

Anhydrous THF (12 ml) was added to 1,3-dimethoxy-5-pentylbenzene (6.66 g, 32 mmol) under argon. A solution of n-butyllithium (20 ml, 1.6 M in hexanes, 32 mmol) was added and the mixture was refluxed at 40° C. for 2 hours. The solvent was removed under reduced pressure and anhydrous THF (20 ml) was added, and the resulting yellow solution was cooled to 0° C. Solid ZnBr₂ (7.22 g, 32 mmol) was added under a flow of argon. The mixture heated to boiling spontaneously. Stirring was continued for 30 minutes. The catalyst PdCl₂(dppf) (0.35 g, 0.48 mmol) was added followed by methyl 2-bromo-4-methylbenzoate 7.10 g, 31 mmol) and the mixture heated at 70° C. for 72 hours. The reaction was quenched with water, followed by ammonium chloride solution. The aqueous layer was extracted with CH₂Cl₂ (3×50 ml) and the combined organic fraction was dried (MgSO₄), filtered and evaporated to dryness. Purification by column chromatography (EA/hexanes, 1:20) gave the product as a crystalline white solid. Yield=7.30 g.

### Example 26

### Preparation of 2-(2′,6′-dimethoxy-5-methyl-4′-pentylbiphenyl-2-yl)propan-2-ol

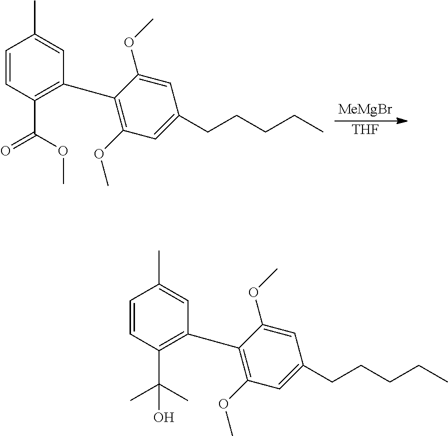 [SMILES: "MeMgBr" ; CCCCCc1cc(OC)c(-c2cc(C)ccc2C(=O)OC)c(OC)c1 ; CCCCCc1cc(OC)c(-c2cc(C)ccc2C(C)(C)O)c(OC)c1 ; "THF"]

A solution of MeMgBr (35 ml, 3 M solution in ether, 105 mmol) was added to a solution of methyl 2′,6′-dimethoxy-5-methyl-4′-pentylbiphenyl-2-carboxylate (7.2 g, 20.2 mmol) in THF (60 ml) under argon and the mixture stirred for 2 hours at room temperature. The reaction was quenched with water, followed by ammonium chloride solution. The aqueous layer was extracted with CH₂Cl₂ (3×50 ml) and the combined organic fraction was dried (MgSO₄), filtered and evaporated to dryness. Yield=7.15 g.

### Example 27

### Preparation of 6,6,9-trimethyl-3-pentyl-6H-benzo[c]chromen-1-ol (cannabinol)

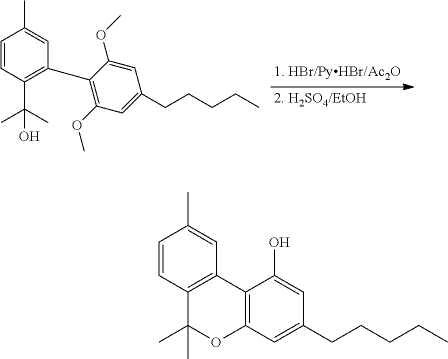 [SMILES: CCCCCc1cc(OC)c(-c2cc(C)ccc2C(C)(C)O)c(OC)c1 ; CCCCCc1cc(O)c2c(c1)OC(C)(C)c1ccc(C)cc1-2]

Pyridinium bromide (13.73 g, 85.6 mmol), hydrobromic acid (48%, 3.48 g, 42.9 mmol) and acetic anhydride (16 ml) were added to 2-(2′,6′-dimethoxy-5-methyl-4′-pentylbiphenyl-2-yl)propan-2-ol (2.7 g, 8.59 mmol) and the mixture was heated at 115° C. for 18 hours. It was cooled to room temperature and diluted with water. It was neutralized with NaOH solution to about pH 6, then extracted with CH₂Cl₂ (3×50 ml). The organic fraction was dried (MgSO₄), then filtered, and evaporated to dryness. The residue was suspended in a mixture of H₂SO₄ (2M, 25 ml) and ethanol (25 ml) and stirred for 18 hours. It was neutralized with NaOH solution to about pH 6, then extracted with CH₂Cl₂ (3×50 ml). The organic fraction was dried (MgSO₄), filtered, and evaporated to dryness. The residue was dissolved in hexanes and filtered through a short pad of silica gel. The filtrate was evaporated to dryness to give the product as a pale-yellow oil. Yield=1.81 g.

### Example 28

### Preparation of 2-(2′,6′-dimethoxy-5-methyl-4′-pentyl-[1,1′-biphenyl]-2-yl)propan-1,1,1,3,3,3-d₆-2-ol

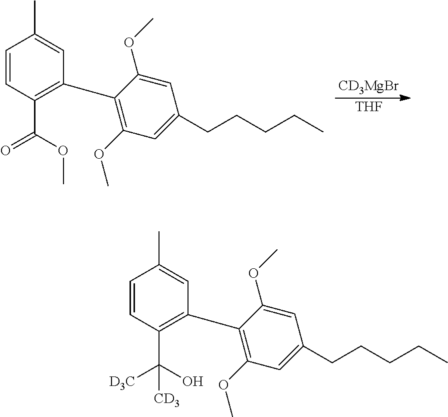 [SMILES: CCCCCc1cc(OC)c(-c2cc(C)ccc2C(=O)OC)c(OC)c1 ; "CD3MgBr" ; [2H]C([2H])([2H])C(O)(c1ccc(C)cc1-c1c(OC)cc(CCCCC)cc1OC)C([2H])([2H])[2H] ; "THF"]

This was prepared using the procedure described in Example 26, using CD₃MgBr.

### Example 29

### Preparation of 2-(2′,6′-dimethoxy-5-methyl-4′-pentyl-[1,1′-biphenyl]-2-yl)propan-2-ol-1,3-¹³C₂

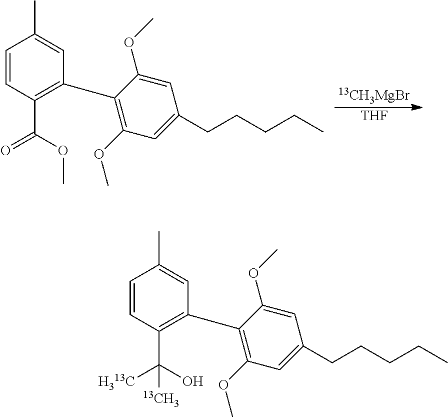

This was prepared using the procedure described in Example 26, using ¹³CH₃MgBr.

While the foregoing disclosure has been described in some detail for purposes of clarity and understanding, it will be appreciated by one skilled in the art, from a reading of the disclosure that various changes in form and detail can be made without departing from the true scope of the disclosure in the appended claims.

All publications, patents, and patent applications are herein incorporated by reference in their entirety to the same extent as if each individual publication, patent or patent application was specifically and individually indicated to be incorporated by reference in its entirety.

1. A compound of Formula (I): 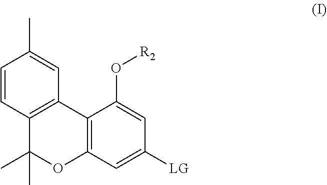
wherein,
LG is any suitable leaving group, and
R₂ represents hydrogen, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

2. The compound of Formula (I) according to claim 1, wherein LG is a sulphonate, a halide, a boronate or MXₙ,
Wherein,
M is Li, Mg, Zn, Sn, B, or Si,
X is halide, OH, or OR, wherein R is (C₁-C₂₀)-alkyl, (C₂-C₂₀)-alkenyl group, a (C₂-C₂₀)-alkynyl group, a (C₃-C₂₀)-cycloalkyl group, or (C₆-C₂₀)-aryl, and
N is O, 1, 2 or 3.

3. The compound of Formula (I) according to claim 1, wherein X is halide, OH, or OR, wherein R is (C₁-C₆)-alkyl, (C₂-C₆)-alkenyl group, a (C₂-C₆)-alkynyl group, a (C₃-C₆)-cycloalkyl group, or (C₆)-aryl group.

4. The compound of Formula (I) according to claim 1, wherein LG is a halide, a sulphonate or a boronate.

5. The compound of Formula (I) according to claim 4, wherein the boronate leaving group is —B(OR)₂, where R is H, a (C₁-C₂₀)-alkyl group, a (C₂-C₂₀)-alkenyl group, a (C₂-C₂₀)-alkynyl group, a (C₃-C₂₀)-cycloalkyl group, or a (C₆-C₁₄)-aryl group.

6. (canceled)

7. The compound of Formula (I) according to claim 4, wherein the sulfonate group is of the formula 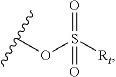
wherein,
Rₜ is a hydrogen atom, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an OR^{c} group or an NR^{c}₂ group, possibly substituted, with possible and non-limiting substituents of Rₜ being halogen atoms, OR^{c}, or NR^{c}₂ groups, in which R^{c} is a hydrogen atom or a cyclic, linear or branched alkyl, aryl or alkenyl group.

8. The compound of Formula (I) according to claim 4, wherein Rₜ is a hydrogen atom, an optionally substituted (C₁-C₁₀)-alkyl group, an optionally substituted (C₂-C₁₀)-alkenyl group, an optionally substituted (C₂-C₁₀)-alkynyl group, an optionally substituted (C₃-C₁₀)-cycloalkyl group, an optionally substituted (C₆-C₁₀)-aryl group.

9. The compound of Formula (I) according to claim 8, wherein the sulfonate group is a triflate group, a mesylate group or a tosylate group.

10. The compound of Formula (I) according to claim 1, wherein R₂ represents a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(′O)—R′, wherein R′ is a (C₁-C₂₀)-alkyl group, wherein each group is each optionally substituted with one or more halogen atoms (F, Cl, Br or I), a (C₁-C₂₀)-alkyl group, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

11. (canceled)

12. A compound of Formula (I) according to claim 1 which is a compound of Formula (II): 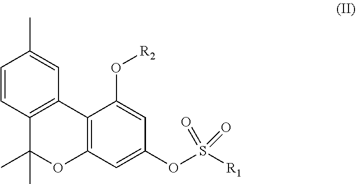
wherein,
R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group;
R₂ is as defined as above for Formula (I) and represents hydrogen, a linear or branched alkyl group of any length, possibly substituted, or an alkenyl group of any length, possibly substituted, or an alkynyl group, possibly substituted, or a cycloalkyl group, possibly substituted, or a alkyl-substituted silyl group, possibly substituted, or an aryl group, possibly substituted, or an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

13. (canceled)

14. A compound of Formula (II) according to claim 12, R₁ is a hydrogen atom, an optionally substituted (C₁-C₆)-alkyl group, an optionally substituted (C₂-C₆)-alkenyl group, an optionally substituted (C₂-C₆)-alkynyl group, an optionally substituted (C₃-C₆)-cycloalkyl group, an optionally substituted (C₆)-aryl group.

15. (canceled)

16. A compound of Formula (II) according to claim 12, R₂ represent a (C₁-C₂₀)-alkyl group, a (C₂-C₂₀)-alkenyl group, a (C₂-C₂₀)-alkynyl group, a (C₃-C₂₀)-cycloalkyl group, a —Si[(C₁-C₂₀)-alkyl]₃ group, a (C₆-C₁₄)-aryl group, or a (C₅-C₁₄)-heteroaryl group, or an acyl group —C(═O)—R′, wherein R′ is a (C₁-C₂₀)-alkyl group, wherein each group is each optionally substituted with one or more halogen atoms (F, Cl, Br or I), a (C₁-C₂₀)-alkyl group, a (C₂-C₂₀)-alkenyl group, a (C₂-C₂₀)-alkynyl group, —OR^{d}, or —NR^{d} 2, wherein R^{c} and R^{d} are independently or simultaneously hydrogen, (C₁-C₂₀)-alkyl, (C₂-C₂₀)-alkenyl, or (C₂-C₂₀)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen (F, Cl, Br or I), or a —(C₁-C₂₀)-alkyl groups.

17. A compound of Formula (II) according to claim 12, wherein R₂ represent a (C₁-C₆)-alkyl group, a (C₂-C₆)-alkenyl group, a (C₂-C₆)-alkynyl group, a (C₃-C₆)-cycloalkyl group, a —Si[(C₁-C₆)-alkyl]₃ group, a phenyl group, or a (C₅-C₆)-heteroaryl group, or an acyl group —C(═O)—R′, wherein R′ is a (C₁-C₆)-alkyl group, wherein each group is each optionally substituted with one or more halogen atoms, a —(C₁-C₆)-alkyl group, a (C₂-C₆)-alkenyl group, a (C₂-C₆)-alkynyl group, —OR^{d}, or —NR^{d}₂, wherein R^{c} and R^{d} are independently or simultaneously hydrogen, (C₁-C₆)-alkyl, (C₂-C₆)-alkenyl, or (C₂-C₆)-alkynyl, and wherein one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more halogen, or a —(C₁-C₆)-alkyl groups.

18. A compound of Formula (II) according to claim 17, wherein R₂ represent a —Si[(C₁-C₆)-alkyl]₃ group. In one embodiment, R₂ represent a —Si[(C₁-C₃)-alkyl]₃ group.

19. A compound of Formula (I) according to claim 1 which is a compound of Formula (III) 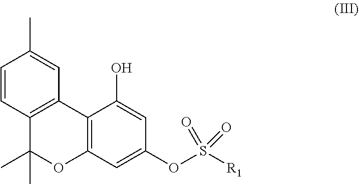
wherein,
R₁ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted, or an heteroaryl group, possibly substituted, or an OR^{c} group or an NR^{c}₂ group, possibly substituted, with possible and non-limiting substituents of R₁ being halogen atoms, OR^{c}, or NR^{c}₂ groups, in which R^{c} is a hydrogen atom or a cyclic, linear or branched alkyl, aryl or alkenyl group.

20. A compound of Formula (III) according to claim 19, wherein R₁ represents a hydrogen atom, —OR^{c}, —NR^{c}₂, fluoro-substituted-(C₁-C₂₀)-alkyl, a (C₁-C₂₀)-alkyl group, a (C₂-C₂₀)-alkenyl group, a (C₂-C₂₀)-alkynyl group, a (C₃-C₂₀)-cycloalkyl group, a (C₆-C₁₄)-aryl group, or a (C₅-C₁₄)-heteroaryl group, wherein the latter 6 groups are each optionally substituted with one or more halogen atoms (F, Cl, Br or I), —(C₁-C₂₀)-alkyl, a (C₂-C₂₀)-alkenyl group, a (C₂-C₂₀)-alkynyl group, —OR^{d}, or —NR^{d}₂, wherein R^{c} and R^{d} are independently or simultaneously hydrogen, (C₁-C₂₀)-alkyl, (C₂-C₂₀)-alkenyl, or (C₂-C₂₀)-alkynyl.

21. (canceled)

22. A compound of Formula (I) according to claim 1, which is

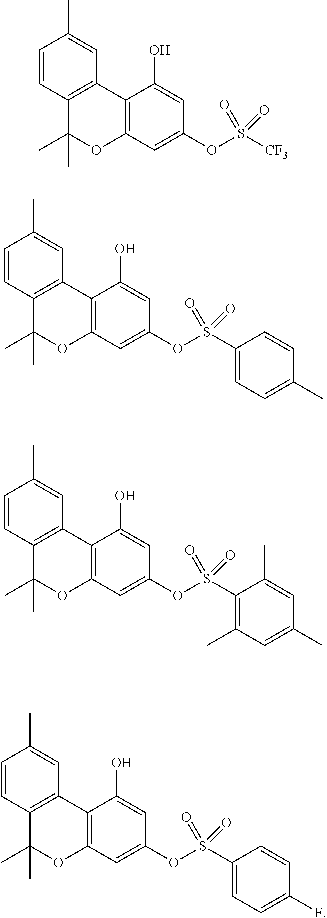 [SMILES: Cc1ccc2c(c1)-c1c(O)cc(OS(=O)(=O)C(F)(F)F)cc1OC2(C)C ; Cc1ccc(S(=O)(=O)Oc2cc(O)c3c(c2)OC(C)(C)c2ccc(C)cc2-3)cc1 ; Cc1cc(C)c(S(=O)(=O)Oc2cc(O)c3c(c2)OC(C)(C)c2ccc(C)cc2-3)c(C)c1 ; Cc1ccc2c(c1)-c1c(O)cc(OS(=O)(=O)c3ccc(F)cc3)cc1OC2(C)C]

23.-45. (canceled)

46. A process for the preparation of compounds of Formula (VII): 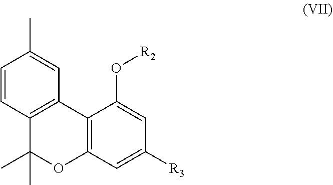
and Formula (VIII): 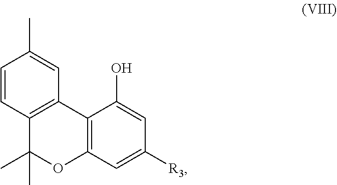
by contacting, in the presence or absence of a catalyst, a compound of Formula (I) as defined in claim 1, with a compound R₃—W, wherein R₃ is a nucleophilic group and W is an electrophilic group, wherein R₃—W is R₃—B(OH)₂, R₃—B(OR)₂, R₃—BF₃K, a R₃—MgX, or R₃—ZnX, wherein X is halide;
R₃ represents a hydrogen atom, a linear or branched alkyl group of any length, possibly substituted, or an alkenyl group of any length, possibly substituted, or an alkynyl group, possibly substituted, or a cycloalkyl group, possibly substituted, or an aryl group, possibly substituted; and
R₂ represents hydrogen, a linear or branched alkyl group of any length, possibly substituted, an alkenyl group of any length, possibly substituted, an alkynyl group, possibly substituted, a cycloalkyl group, possibly substituted, an aryl group, possibly substituted, an heteroaryl group, possibly substituted, or an acyl group, possibly substituted, and one or more of the carbon atoms in the alkyl, alkenyl, alkynyl, cycloalkyl, aryl, heteroaryl or acyl groups of R₂ is optionally replaced with a heteroatom selected from the group consisting of O, S, N, P and Si, which, where possible, is optionally substituted with one or more groups.

47. A process according to claim 46, involving catalytic and non-catalytic carbon-carbon bond forming reactions.

48. A process according to claim 47, wherein the bond forming reactions include Ullman, Suzuki-Miyaura, Negishi, Kumada, Sonogashira and Stille reactions.
